(19) United States
(12) Patent Application Publication
(10) Pub. No.: US 2022/0211633 A1
(43) Pub. Date: Jul. 7, 2022

(54) HIGH DENSITY LIPOPROTEIN NANOPARTICLES AND RNA TEMPLATED LIPOPROTEIN PARTICLES FOR OCULAR THERAPY

(71) Applicant: Northwestern University, Evanston, IL (US)

(72) Inventors: C. Shad Thaxton, Chicago, IL (US); Robert M. Lavker, Evanston, IL (US); Kaylin M. McMahon, Chicago, IL (US); Han Peng, Evanston, IL (US); Andrea E. Calvert, Chicago, IL (US); Nihal Kaplan, Evanston, IL (US)

(73) Assignee: Northwestern University, Evanston, IL (US)

(21) Appl. No.: 17/605,510

(22) PCT Filed: Apr. 24, 2020
(86) PCT No.: PCT/US2020/029752

§ 371 Date: Oct. 21, 2021

Related U.S. Application Data
(60) Provisional application No. 62/839,579, filed on Apr. 26, 2019.

(51) Int. Cl. A61K 9/51; A61K 31/713; A61K 9/16

(52) U.S. Cl. CPC A61K 9/5123; A61K 9/1617; A61K 9/5115; A61K 31/713

(57) ABSTRACT

Disclosed herein are nanostructures, compositions, and methods for treating ocular disorders, injuries, and infections using RNA complexed nanoparticles (e.g., RNA-templated lipoprotein particles, miRNA-high density lipoprotein particles). These nanostructures are contemplated in topical therapies.

### CROSS-REFERENCE TO RELATED APPLICATIONS

This application claims the benefit under 35 U.S.C. § 119(e) of the filing date of U.S. Patent Application Ser. No. 62/839,579, filed Apr. 26, 2019. The contents of the above-referenced application is hereby incorporated herein in its entirety by reference.

### GOVERNMENT SUPPORT

This invention was made with government support under R01 EY019463 and R01 CA167041, both awarded by the National Institutes of Health (NIH). The government has certain rights in the invention.

### BACKGROUND OF THE INVENTION

Ocular disorders (eye diseases), infections, and injuries are challenging to treat and, if left untreated can have devastating effects on patients (e.g., irreparable damage, blindness, etc.). For example, diabetes mellitus cornea is the leading cause of legal blindness. Patients with diabetes mellitus can develop proliferative diabetic retinopathy (PDR), and those with PDR lose their vision often within 5 years (43% and 60%, Type 1 and 2, respectively). Of these patients, up to 70% have corneal problems. Such problems may manifest, for example, as increased corneal thickness; epithelial defects, fragility, and erosion; ulcers; edema; superficial punctate keratitis; endothelial changes; neuropathy; and delayed and/or incomplete wound repair. Further complicating these issues, many ocular diseases have no early symptoms, which increases the need for highly effective treatments once they (e.g., ocular disorders) are diagnosed. Due to the frequent ineffectiveness of conventional treatments for ocular disorders, infections, and injuries, there is an increasing need for improved therapies.

### SUMMARY OF THE INVENTION

The present disclosure presents compositions and methods for treating diseases or injuries of the eye (e.g., the anterior ocular segment (e.g., cornea, limbus, and conjunctiva)). Treatments for these regions face multiple barriers to effectiveness. For example, the eye comprises a variety of physical barriers (e.g., tear film, lipid layers, aqueous layers, mucus layers, epithelial layers, and cellular layers (e.g., stroma, etc.) as well as mechanical barriers (e.g., blink reflex). Accordingly, the present disclosure presents new compositions which can overcome these problems to deliver compositions for treatment.

The present disclosure is based, at least in part, on compositions or methods of using RNAs (e.g., miRNAs) bound to nanostructures (e.g., high density lipoproteins (HDL-NPs) or templated lipoprotein particles (TLPs) to treat (e.g., topically) diseases or injuries of the anterior ocular segment (e.g., cornea, limbus, and conjunctiva).

Accordingly, one aspect of the present disclosure provides a nanostructure, comprising a high density lipoprotein nanoparticle (HDL-NP) comprising a core, an apolipoprotein, a lipid shell attached to the core, wherein the lipid shell comprises a phospholipid and an RNA molecule that is associated with the phospholipid. Another aspect of the present disclosure provides a nanostructure comprising a templated lipoprotein particle (TLP) comprising a core, an apolipoprotein, a lipid shell attached to the core, wherein the lipid shell comprises a phospholipid and an RNA molecule that is associated with the phospholipid. In some embodiments, the apolipoprotein in the nanostructure is apolipoprotein A-I (also as may be referred to herein as apoA-I, A-1, or AI). In some embodiments, the nanostructure further comprises a cholesterol.

Another aspect of the present disclosure provides a method of treating a subject having an ocular disorder, comprising administering at least one of the nanostructures as described herein to the subject in an effective amount, thereby treating the ocular disorder.

Another aspect of the present disclosure provides a method of treating a subject having an ocular injury or ocular infection, comprising administering at least one of the nanostructures as described herein to the subject in an effective amount, thereby treating the ocular injury or infection. In some embodiments, the ocular disorder, ocular injury, or ocular infection is a corneal disorder, corneal injury, or corneal infection, respectively. In some embodiments, the ocular disorder is diabetic keratopathy. In some embodiments, the administration of the nanostructure is by means of topical administration.

In some embodiments of the present disclosure, the RNA molecule is a microRNA (miRNA). In some embodiments, the miRNA is miR-205 or miR-146a.

An anionic nanostructure is provided in other aspects of the invention. The anionic nanostructure comprises an aggregate of cationic lipid-RNA complexes and a templated lipoprotein particle (TLP) wherein the TLP comprises an anionic TLP which is a synthetic HDL having an inert core, a lipid shell surrounding the inert core, and an apolipoprotein functionalized to the inert core, wherein the RNA molecule is a microRNA (miRNA) and wherein the aggregate of cationic lipid-nucleic acid complexes and TLPs forms the anionic nanostructure aggregate.

In some embodiments the cationic lipid-nucleic acid complex is comprised of single stranded miRNA complexed with the cationic lipid. In some embodiments the miRNA is miR-205 or miR-146a. In some embodiments the aggregate of cationic lipid-nucleic acid complexes and TLPs has a negative ζ-potential. In some embodiments n the aggregate of cationic lipid-RNA comprises a mixture of cationic lipid-sense strand RNA and cationic lipid-antisense strand RNA. In some embodiments the RNA is not chemically modified. In some embodiments the RNA is chemically modified. In some embodiments the phospholipids are selected from 1,2-dioleoyl-sn-glycero-3-phophocholine (DOPC) and 1,2-dipalmitoyl-sn-glycero-3-phosphoethanolamine-N-[3-(2-pyridyldithio)propionate] (PDP-PE). In some embodiments the nanostructure comprises alternating layers of 1,2-dioleoyl-3-trimethylammonium-propane (DOTAP) and miRNA.

Another aspect of the present disclosure provides a pharmaceutical composition comprising any one of the nanostructures as described herein, or any combination of the nanostructures disclosed herein.

In some aspects, the disclosure relates to a method of treating a subject having ocular inflammation, comprising: administering the nano structure of any one of the nanostructures of the disclosure to the subject in an effective amount, thereby treating the ocular inflammation.

In some aspects, the disclosure relates to a method of inhibiting NF_{K}B signaling in a subject having, comprising: administering the nanostructure of any one of the nanostructures of the disclosure to the subject in an effective amount, wherein the RNA is miRNA and wherein the miRNA is miR-146a.

In some embodiments, the nanostructures of the disclosure are used to treat a subject. In some embodiments, the subject is a mammal. In some embodiments, the subject is human.

These and other aspects and embodiments will be described in greater detail herein. The description of some exemplary embodiments of the disclosure are provided for illustration purposes only and not meant to be limiting. Additional compositions and methods are also embraced by this disclosure.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, Drawings, Examples, and Claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. For purposes of clarity, not every component may be labeled in every drawing. It is to be understood that the data illustrated in the drawings in no way limit the scope of the disclosure. In the drawings:

FIGS. 1A-1B show synthetic spherical HDL-NPs (FIG. 1A) and a comparison of properties of native HDL and synthetic HDL-NPs (FIG. 1B).

FIGS. 2A-2C show templated lipoprotein particle (TLP) synthesis (FIG. 2A) and structures of CL:Cardiolipin (FIG. 2B) and 18:2 PG (FIG. 2C).

FIGS. 3A-3B show two different schematics of scavenger receptor B1 (SR-B1) as a means of TLP transport (FIGS. 3A-3B).

FIGS. 4A-4D show SR-B1 is expressed on corneal epithelial cells. Immunofluorescence (IF) staining of human cornea (FIG. 4A), murine cornea (FIG. 4B), and murine limbus (FIG. 4C) shows SR-B1 expression in the epithelial cells and in the stroma (arrows). Human corneal epithelial cells (HCECs) express SR-B1 protein as seen by western blot (FIG. 4D).

FIG. 5 includes images of human corneal epithelial cells (HCECs) and high density lipoprotein nanoparticles (HDL-NPs) accumulating in the cytoplasm of the cells.

FIGS. 6A-6F show a schematic of Akt mitigated wound healing pathway (FIG. 6A), Absorbance results of miR-205 AI NP synthesis by method of FIG. 2A (FIG. 6B), SHIP2 protein expression is decreased in human corneal epithelial cells when treated with miR-205-AI particles as seen by western blot analysis (FIG. 6C) and quantified by densitometry (FIG. 6D), Phospho-Akt protein expression is increased in human corneal epithelial cells when treated with miR-205-AI particles as seen by western blot analysis (FIG. 6E), and that miR-205 HDL-NPs decreased SHIP2 and increased p-Akt after treatment (FIG. 6F).

FIG. 7 shows miR-205-HDL-NPs rapidly sealing scratch wounds.

FIG. 8 includes a plot showing miR-205-HDL-NPs rapidly sealing scratch wounds compared to control (Nanoparticle-NC-miR).

FIG. 9 includes a plot showing miR-146 reducing NF-kB activity.

FIG. 10 includes apotome optical sections. 1 μM Cy-3 control RNA-TLP was applied to the murine eye every 30 minutes for 4 hours total. 24 hours after first application of TLP, mice were sacrificed, eyes excised, mounted in OCT and sectioned. Slides were stained for Cy3 (RNA-TLP-red), Keratin 12 (epithelia-green), and DAPI (nucleus-blue).

FIGS. 11A-G include fluorescent microscopy sections of HDL-NP (FIG. 11A) and Cy3-HDL-NP (FIG. 11B) treatment on intact non-wounded corneas; Cy3-labeled AI are detected in corneal epithelial basal (B), wing (W), superficial (S) cells and keratinocytes (K) from healthy murine eyes (FIG. 11C: untreated; FIG. 11D: Cy3-Al NP); and Cy3-labeled AI are detected in corneal epithelial basal (B), wing (W), superficial (S) cells and keratinocytes (K) from wounded murine eyes (FIG. 11E: untreated; FIG. 11F: Cy3-Al NP); and Cy3-labeled AI are detected in the conjunctiva of the eye after wounding (FIG. 11G).

FIGS. 12A-D include diagrams showing that HDL-NPs and miR-205-HDL-NPs exhibit biological activity in vivo (FIGS. 12A-12D). FIG. 12A includes images of such, captured over 24 hours. FIG. 12B includes a plot showing % of wound closure over time. Diet-induced obesity (DIO) were anesthetized and a 1 mm wound in corneal epithelium was made using diamond burr, mice received topical application of miR-205-AI or Scramble-miR-AI every 30 minutes for 2 hours, mice were monitored up to 24 hours post wounding (FIGS. 12C-12D). miR-205-AI and NC-miR-AI both enhance corneal wound healing in DIO mice compared to PBS as seen with fluorescein dye (FIG. 12C); DIO mice have inhibited corneal wound healing compared to mice on a normal diet (ND), AI NPs with or without NC-miR or miR-205 conjugated to the particles reduce wound healing to the same degree in DIO mice (FIG. 12D).

FIGS. 13A-13C show that miR-205-TLP induces p-Akt and reduces SHIP2 protein expression and Al NP increase p-Akt, pEphA2, and DSG3 in corneal epithelial cells as well as that Akt signaling is needed for enhanced wound closure. hTCEpi, hTERT immortalized human corneal epithelial cells, were treated with RNA-TLPs conjugated with either antisense plus sense strands (double strands) or twice the amount of antisense strands (single strand) of miR-205 or a negative control. Lanes to the left show non-treated (NT) cells, negative precursor transfection control, and miR-205 transfection controls (FIG. 13A). AI NP increase phospho-Akt, phospho-EphA2, and DSG3 in human corneal epithelial cells compared to PEG-NPs (FIG. 13B). Human corneal epithelial cells treated with AI NP have enhanced scratch wound closure compared to PEG NP which is abrogated by the PI3K/Akt inhibitor LY294002 (FIG. 13C)

FIGS. 14A-14E show that RNA-TLPs penetrate wounded corneal epithelium; that Al NPs increase F-actin at the leading edge of corneal epithelial scratch wounds; and that inhibition of Ephrin-A1 and activation of Src are needed for Al NP wound closure. A ˜1 mm diameter corneal abrasion wound was made on the cornea of mice. 1 μM Cy3-control-RNA-TLP was topically applied to the eye every 30 minutes for 4 hours. 24 hours post-wounding, eye was excised, mounted in OCT and sectioned. Slides were stained for Cy3 (RNA-TLP-red), Keratin 12 (epithelia-green), and DAPI (nucleus-blue) (FIG. 14A). Human corneal epithelial cells treated with AI NP have enhanced F-actin at the leading edge of scratch wounds (FIG. 14B: PEG-NP; FIG. 14C: HDL-NP). Human corneal epithelial cells treated with AI NP have enhanced scratch wound closure compared to PEG NP which is abrogated by overexpression of Ephrin-A1 (FIG. 14D) or an inhibitor of Src (pp2) (FIG. 14E).

FIG. 15 shows that RNA-TLP penetrate wounded skin. A punch wound was made on the flank of mice. 1 μM Cy3-control-RNA-TLP was topically applied to the wound every 30 minutes for 4 hours. 24 hours post-wounding, skin was excised, mounted in OCT (optimal cutting temperature compound) and sectioned. Slides were stained for Cy3 (RNA-TLP-red), Keratin 15 (basal keratinocytes-green), Keratin 10 (epidermal keratinocytes-white) and DAPI (nucleus-blue).

FIGS. 16A-16G show miR-146a acting on a NF_{K}B signaling pathway (FIG. 16A); miR-146a-TLP inhibit LPS induced NF-κB Signaling (FIG. 16B-16C), J774-Dual mouse macrophage cells were pre-treated with 0.5 ng/mL LPS (O111:B4) for 1 hour, followed by treatment of 40 nM miR-146a-TLP, Ctrl-TLP, or TLP alone, or with lipofectamine delivered miR-146a or control miRNA for 24 hours. QUANTI-Blue assay (InVivoGen) was used to determine NF-κB SEAP (secreted embryonic alkaline phosphatase) activity; Eyes treated with PBS or PEG NP did not have clearing of inflammation of the cornea 7 days post injury, however AI NP had significantly reduced inflammation of the eye (FIGS. 16D-16E); H&E stains of the cornea of eyes treated with PEG NP or AI NP for 7 days following injury show enhanced clearance of inflammation in AI NP treated eyes compared to PEG NP treated eyes. (FIG. 16F); and 3 days post-injury, cornea treated with AI NP had a significant reduction in inflammatory cytokines (IL1a, IL1b, IL6, iNOS, MMP9, and CCL2) (FIG. 16G).

FIG. 17 includes a UV-visible spectra of miR-205-TLP. miR-205-TLP and NC-TLPs have expected UV-visible spectra with a peak at 520 nm (AuNP) and at 260 nm, demonstrating the presence of RNA on the TLPs.

FIG. 18 includes a UV-visible spectra of miR-146a-TLP, miR-146a-TLP and Ctrl-TLP have UV-visible spectra with peaks at 520 nm (Au NP) and at 260 nm (RNA) demonstrating the presence of RNA on the TLPs.

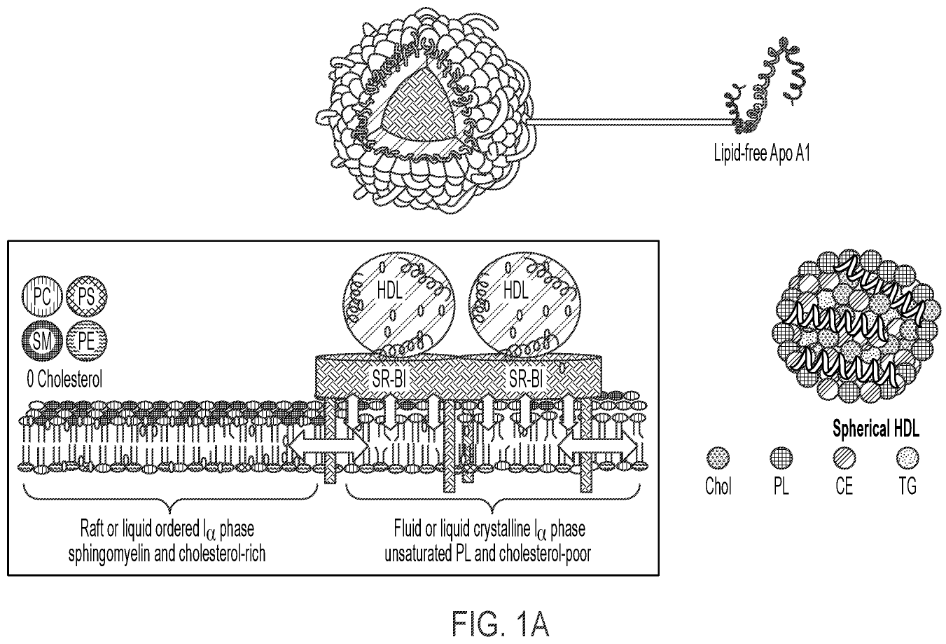

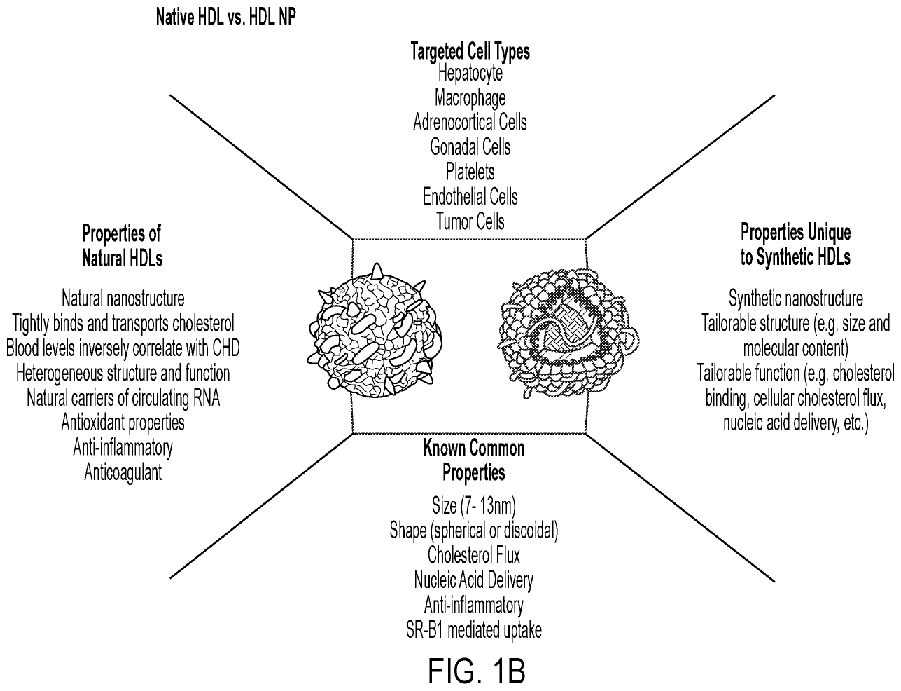

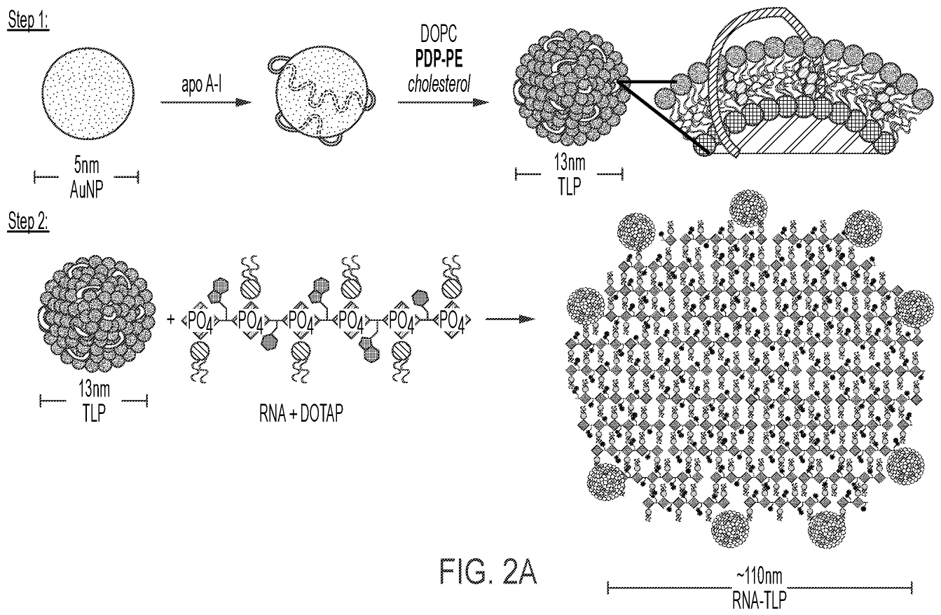

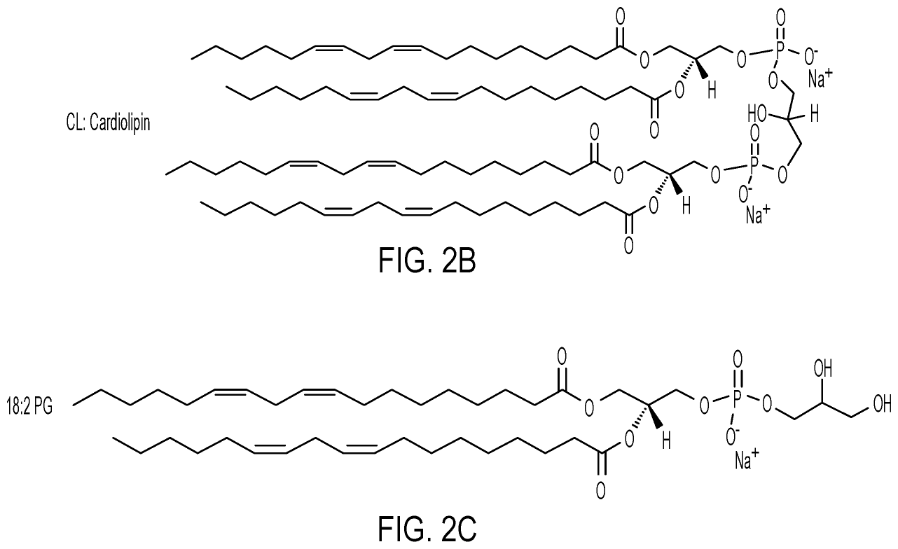

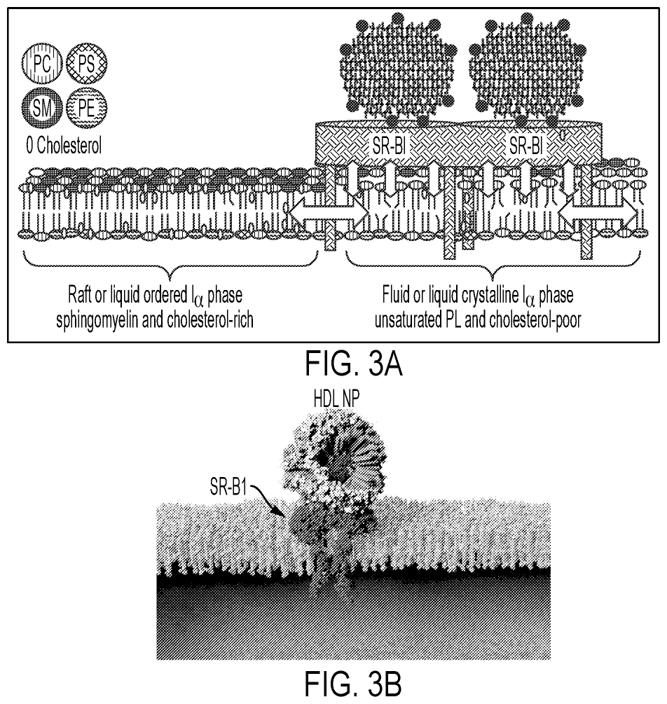

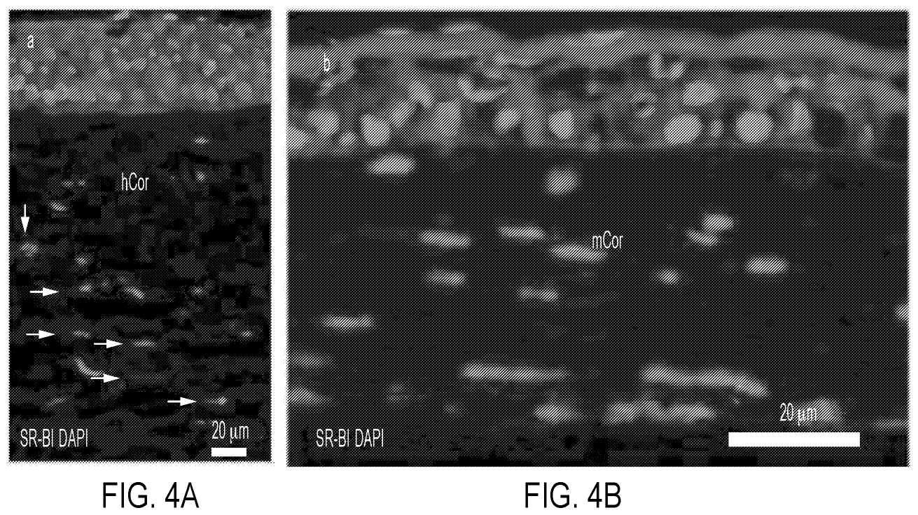

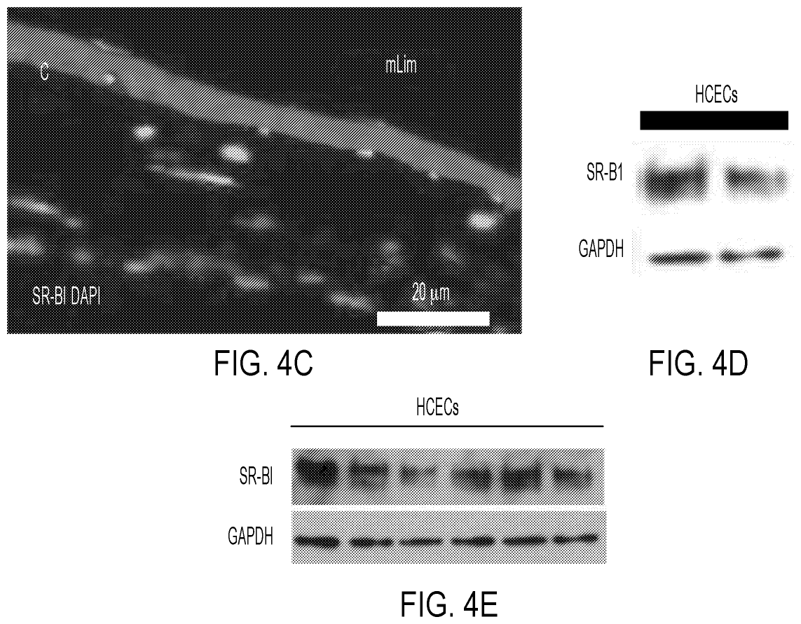

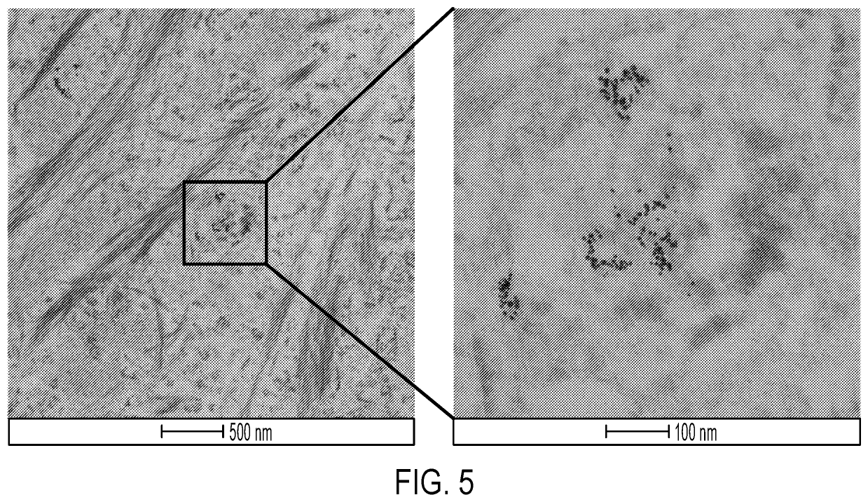

### DETAILED DESCRIPTION OF THE INVENTION

The present disclosure provides compositions or methods of using RNAs (e.g., microRNAs (miRNAs)) bound to nanostructures (e.g., high density lipoproteins (HDL-NPs) or templated lipoprotein particles (TLPs) to treat (e.g., topically) diseases or injuries of the anterior ocular segment (e.g., cornea, limbus, and conjunctiva). In some embodiments, the nanostructures of the present disclosure are used for prophylactic treatment of ocular diseases.

Delivery of therapies to the eye through eye drops faces many challenges including ocular barriers (e.g., tear film and cellular layers), rapid clearance from the eye, and turnover leading to low amounts of drug delivered to the cornea. The anterior surface epithelium, in conjunction with the tear film provides an efficient barrier to the external environment and contributes to the maintenance of corneal transparency and rigidity. While such a barrier is essential for the health of the eye, paradoxically it can prevent delivery of drugs necessary to combat various disease states, such as inflammation and infections. Delivery is further compounded by the blink reflex, which in addition to removing debris and microorganisms from the ocular surface, can also remove topically applied medications. MicroRNAs (miRNAs) are short (˜22 nucleotides in length), “non-coding” or “non-messenger” RNAs that are part of the RNA interference (RNAi) silencing machinery. miRNAs modulate biological homeostasis by controlling gene expression through mRNA targeting and translational repression. As such they contribute to the regulation of a wide variety of biological processes in both normal and disease situations. Consequently, miRNAs hold great promise as potential therapeutic agents. A major hurdle to achieving this goal has been to effectively formulate and deliver therapeutic miRNAs to the cytoplasm of target cells in a stable form. Previous miRNA-related eye treatments have not been delivered topically due to these challenges.

High-density lipoproteins (HDL) are natural in vivo RNA delivery vehicles. Natural high-density lipoproteins (HDLs), isolated from human serum, were found to contain miRNAs and these HDL-bound miRNAs were found to have improved stability compared to naked miRNAs. Additionally, native HDLs deliver bound miRNAs to cells that express the high-affinity scavenger receptor type B-1 (SCARB1) receptor of HDLs. SCARB1 is expressed on corneal epithelial cells.

Herein it was found that the use of spherical, functional, HDL-like nanoparticles (HDL-NP) that can deliver RNA (e.g., miRNAs) topically to the eye, preferably the cornea, has a positive effect on wound healing in diabetic mouse corneas. The HDL-NPs not only transport endogenous miRNAs, which can differ with disease states, but can also deliver miRNAs to recipient cells with functional gene regulatory consequences (e.g., affect expression).

Inspired by features of HDL, templated lipoprotein particles (TLP) were developed that self-assemble with single-strand and single-strand complements of RNA duplex pairs after formulation with a cationic lipid. The resulting RNA templated lipoprotein particles (RNA-TLP) are anionic and tunable with regard to RNA assembly and function. Data show miRNA-205 (miR-205)-TLP actively target and downregulate miR-205, target SHIP-2, and increase phosphorylated-Akt (p-Akt) in a corneal epithelial cell line. In vivo, topical administration to the eye of TLPs conjugated with a non-targeting RNA sequence modified with a Cy3 fluorophore demonstrates penetration of Cy3-labeled RNA in the corneal epithelium, particularly in the basal cells and keratocytes with uptake in the limbal epithelium and stroma. This is a modular approach to topical RNA-delivery to the eye by self-assembling single-strand complements of RNA into actively targeted anionic delivery vehicles that potently regulate target gene expression in vitro and penetrate the corneal epithelium in vivo.

The RNA-templated lipoprotein particles (RNA-TLPs) contemplated herein are a combination of synthetic bio-inspired lipoproteins and cationic lipid-RNA assemblies. They carry the advantage of controlled self-assembly and the functional tunability of RNA-TLPs. Furthermore, the modular nature of the RNA-TLPs (like the HDL-NPs) allow easy exchange of therapeutic RNA cargo, active cell targeting, potent target gene regulation, and in vivo efficacy after ocular administration.

In some embodiments, the process of synthesizing the RNA-TLPs includes surface-functionalization of a solid particle such as a 5 nanometer (nm) diameter gold nanoparticle (Au NP) template with apolipoprotein A-I (apoA-I), a mixture of two phospholipids, and cholesterol. The outer phospholipid and cholesterol favorably associate with nucleic acids. During the synthesis process, due to the negative charge of TLPs and RNA, a cationic lipid (e.g., DOTAP) known to complex RNA, is added to mixtures of RNA in water or phosphate buffered saline (PBS). TLPs mixed with e.g., DOTAP-RNA in PBS become irreversibly aggregated, and precipitate.

Nearly all of the technologies developed for ocular delivery of RNA are based upon cationic lipids or cationic polymers. Most often due to the cationic nature of these vehicles and the synthetic properties, they can be highly toxic and are not typically targeted to disease specific sites. The compositions of the present invention overcome many of these barriers to ocular RNA therapy, because the nanostructures are formulated such that they are anionic and inherently targeted through specific receptors located on the surface of cells.

Many RNA therapies are designed around specific disease targets, however, the nanostructures disclosed herein are highly modular, such that they can be tailored to incorporate presumably any one or multiple target(s) of interest.

Pre-existing techniques are not easily scaled and have unknown biological composition, which can lead to in vivo toxicity. In contrast, the nanostructures disclosed herein have been demonstrated in vivo to have no inherent toxicity and are formulated to mimic natural RNA delivery vehicles to circumvent vehicle related toxicity.

Nanostructures

In some aspects, the disclosure relates to a nanostructure, comprising: a high density lipoprotein nanoparticle (HDL-NP) comprising a core, an apolipoprotein, a lipid shell attached to the core, wherein the lipid shell comprises a phospholipid and an RNA molecule that is associated with the phospholipid.

As used herein, the term “nanostructure” refers to a high density lipoprotein-like nanoparticle (HDL-NP) or a templated lipoprotein particle (TLP), which can be combined with nucleic acids. The nanostructures of the present disclosure are contemplated as being complexed with RNA molecules (e.g., miRNA). As used herein, the terms “HDL-NPs” and “HDL-like nanoparticles” are used interchangeably. High-density lipoproteins (HDL) are native circulating nanoparticles that carry cholesterol, target specific cell types, and play important roles in a host of disease processes. As a result, synthetic HDL mimics have become promising therapeutic agents. However, approaches to date have been unable to reproduce key features of spherical HDLs, which are the most abundant HDL species, and are of particular clinical importance. As used herein, the term “associated” is used to refer to the lipid in the nanostructure being complexed with the lipid. As used herein, the terms “complexed” and “bound” are used interchangeably.

In some aspects, the disclosure relates to a nanostructure comprised of a templated lipoprotein particle (TLP) comprising a core, an apolipoprotein, a lipid shell attached to the core wherein the TLP is complexed to an RNA molecule through a cationic lipid. A TLP, in some embodiments forms an anionic nanostructure aggregate with RNA. The nanostructure comprises an aggregate of cationic lipid-nucleic acid complexes and templated lipoprotein particles (TLP), wherein the TLP comprises an anionic TLP which is a synthetic HDL having an inert core, a lipid shell surrounding the inert core, and an apolipoprotein functionalized to the inert core; and the cationic lipid-nucleic acid complex, is comprised of single stranded or double stranded RNA complexed with a cationic lipid, and wherein the aggregate of cationic lipid-nucleic acid complexes and TLPs has a negative ζ-potential and forms the anionic nanostructure aggregate. In some embodiments each strand of a duplex RNA is conjugated separately to a cationic lipid. In some embodiments the RNA is not chemically modified. In other embodiments it is chemically modified. In some embodiments the inert core is a metal such as gold. In some embodiments the phospholipids are 1,2-dioleoyl-sn-glycero-3-phophocholine (DOPC) and 1,2-dipalmitoyl-sn-glycero-3-phosphoethanolamine-N-[3-(2-pyridyldithio)propionate] (PDP-PE). In some embodiments the nanostructure comprises alternating layers of 1,2-dioleoyl-3-trimethylammonium-propane (DOTAP) and RNA.

In some embodiments, the nanostructure includes a cationic lipid. The cationic lipid may be, for example, N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.C1), 1,2-Dilinoleoyl-3-trimethylaminopropane chloride salt (DLin-TAP.C1), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3 aH-cyclopenta[d][1,3]dioxol-5-amine, (6Z,9Z,28Z,31Z)-heptatriaconta-6,9,28,31-tetraen-19-yl-4-(dimethylamino)butanoate, or a mixture thereof.

Other cationic lipids, which carry a net positive charge at about physiological pH, in addition to those specifically described above, may also be included in the lipid nanoparticle. Such cationic lipids include, but are not limited to, N,N-dioleyl-N,N-dimethylammonium chloride (“DODAC”); N-(2,3-dioleyloxy)propyl-N,N—N-triethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 1,2-Dioleyloxy-3-trimethylaminopropane chloride salt (“DOTAP.Cl”); 3.beta.-(N—(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethyl-ammonium trifluoracetate (“DOSPA”), dioctadecylamidoglycyl carboxyspermine (“DOGS”), 1,2-dileoyl-sn-3-phosphoethanolamine (“DOPE”), 1,2-dioleoyl-3-dimethylammonium propane (“DODAP”), N,N-dimethyl-2,3-dioleyloxy)propylamine (“DODMA”), N-(1,2-dimyristyloxyprop-3-yl)-N,N-dimethyl-N-hydroxyethyl ammonium bromide (“DMRIE”), and 1,2-dioleoyl-sn-glycero-3-phosphocholine (“DOPC”).

In some aspects of the disclosure, the nanostructure comprises a cationic lipid (e.g., DOTAP) is mixed with a nucleic acid (e.g., RNA) in a molar ratio of about 1:1, of about 2:1, of about 3:1, of about 4:1, of about 5:1, of about 6:1, of about 7:1, of about 8:1, of about 9:1, of about 10:1, of about 11:1, of about 12:1, of about 13:1, of about 14:1, of about 15:1, of about 16:1, of about 17:1, of about 18:1, of about 19:1, of about 20:1, of about 21:1, of about 22:1, of about 23:1, of about 24:1, of about 25:1, of about 26:1, of about 27:1, of about 28:1, of about 29:1, of about 30:1, of about 31:1, of about 32:1, of about 33:1, of about 34:1, of about 35:1, of about 36:1, of about 37:1, of about 38:1, of about 39:1, of about 40:1, of about 41:1, of about 42:1, of about 43:1, of about 44:1, of about 45:1, of about 46:1, of about 47:1, of about 48:1, of about 49:1, of about 50:1, of about 60:1, of about 70:1, of about 80:1, of about 90:1, or of about 100:1. In some embodiments, the cationic lipid (e.g. DOTAP) is mixed with the nucleic acid (e.g., RNA) in a molar ratio of 10:1, 20:1, 30:1 or 40:1.

“Amphipathic lipids” refer to any suitable material, wherein the hydrophobic portion of the lipid material orients into a hydrophobic phase, while the hydrophilic portion orients toward the aqueous phase. Such compounds include, but are not limited to, phospholipids, aminolipids, and sphingolipids. Representative phospholipids include sphingomyelin, phosphatidylcholine, phosphatidylethanolamine, phosphatidylserine, phosphatidylinositol, phosphatidic acid, palmitoyloleoyl phosphatdylcholine, lysophosphatidylcholine, lysophosphatidylethanolamine, dipalmitoylphosphatidylcholine, dioleoylphosphatidylcholine, distearoylphosphatidylcholine, or dilinoleylphosphatidylcholine, monophosphoryl lipid A (MPLA), or glycopyranoside lipid A (GLA).

In some embodiments, the nanostructures of the disclosure comprise apolipoprotein. The apolipoprotein can be apolipoprotein A (e.g., apo A-I, apo A-II, apo A-IV, and apo A-V), apolipoprotein B (e.g., apo B48 and apo B100), apolipoprotein C (e.g., apo C-I, apo C-II, apo C-III, and apo C-IV), and apolipoproteins D, E, and H. Additionally, a structure described herein may include one or more peptide analogues of an apolipoprotein, such as one described above. Of course, other proteins (e.g., non-apolipoproteins) can also be included in the nanostructures described herein. In some embodiments, the nanostructure of the present disclosure contain apolipoprotein A-I (apoA-I), which is the main protein constituent of HDLs. The nanostructures of the present disclosure are able to bind with high affinity to SCARB1. The nanostructures of the present disclosure have reduced toxicity. In some embodiments, the apolipoprotein is apolipoprotein A-I.

The nanostructures of the present disclosure are used for treatment of diseases, infections, and injuries. Disorders, infections and injuries that are contemplated herein include, without limitation, corneal injury, dry-eye, keratitis, conjunctivitis, cataract, glaucoma, eye inflammation, uveitis, and iritis.

The surface density of bound oligonucleotides to the structures may also be controlled. Oligonucleotides such as DNA, RNA, or siRNA may be attached to a nanostructure core using techniques such as electrostatic adsorption or chemisorption techniques, for example, Au—SH conjugation chemistry.

High Density Lipoprotein Nanoparticles (HDL NPs) Core

The core of the nanostructure may be hollow or a nanostructure core. The core of the nanostructure whether being a nanostructure core or a hollow core, may have any suitable shape and/or size. For instance, the core may be substantially spherical, non-spherical, oval, rod-shaped, pyramidal, cube-like, disk-shaped, wire-like, or irregularly shaped. In some embodiments, the core comprises a substantially spherical shape. In some embodiments, the core comprises a substantially non-spherical shape. In some embodiments, the core comprises a substantially oval shape. In some embodiments, the core comprises a substantially rod-like shape. In some embodiments, the core comprises a substantially pyramidal shape. In some embodiments, the core comprises a substantially cube-like shape. In some embodiments, the core comprises a substantially disk-like shape. In some embodiments, the core comprises a substantially wire-like shape. In some embodiments, the core comprises a substantially irregular shape. The core (e.g., a nanostructure core or a hollow core) may have a largest cross-sectional dimension (or, sometimes, a smallest cross-section dimension) of, for example, less than or equal to about 500 nm, less than or equal to about 250 nm, less than or equal to about 100 nm, less than or equal to about 75 nm, less than or equal to about 50 nm, less than or equal to about 40 nm, less than or equal to about 35 nm, less than or equal to about 30 nm, less than or equal to about 25 nm, less than or equal to about 20 nm, less than or equal to about 15 nm, or less than or equal to about 5 nm. In some cases, the core has an aspect ratio of greater than about 1:1, greater than 3:1, or greater than 5:1. As used herein, “aspect ratio” refers to the ratio of a length to a width, where length and width measured perpendicular to one another, and the length refers to the longest linearly measured dimension.

The core may be formed of an inorganic material. The inorganic material may include, for example, a metal (e.g., Ag, Au, Pt, Fe, Cr, Co, Ni, Cu, Zn, and other transition metals), a semiconductor (e.g., silicon, silicon compounds and alloys, cadmium selenide, cadmium sulfide, indium arsenide, and indium phosphide), or an insulator (e.g., ceramics such as silicon oxide). In some embodiments, the core is gold (Au). The inorganic material may be present in the core in any suitable amount, e.g., at least 1 percent by weight (i.e., 1 wt %), 5 wt %, 10 wt %, 25 wt %, 50 wt %, 75 wt %, 90 wt %, or 99 wt %. In one embodiment, the core is formed of 100 wt % inorganic material. The nanostructure core may, in some cases, be in the form of a quantum dot, a carbon nanotube, a carbon nanowire, or a carbon nanorod. In some cases, the nanostructure core comprises, or is formed of, a material that is not of biological origin. In some embodiments, a nanostructure includes or may be formed of one or more organic materials such as a synthetic polymer and/or a natural polymer. Examples of synthetic polymers include non-degradable polymers such as polymethacrylate and degradable polymers such as polylactic acid, polyglycolic acid, and copolymers thereof. Examples of natural polymers include hyaluronic acid, chitosan, and collagen. In certain embodiments, the structure, nanostructure or nanoparticle core does not include a polymeric material (e.g., it is non-polymeric).

In some embodiments, the structure, nanostructure, or nanoparticle disclosed herein has 60-250 fold molar excess lipid to gold core. In some embodiments, the structure, nanostructure, or nanoparticle disclosed herein has 60-200, 60-150, 60-100, 60-75, 70-200, 70-150, 70-100, 70-75, 80-250, 80-200, 80-150, 80-100, 90-250, 90-200, 90-150, 90-100, 100-250, 100-200, 100-150, 62.5, 125, 187.5, or 250 fold molar excess lipid to the core (e.g., gold core).

High Density Lipoprotein Nanoparticles (HDL NPs) Shell

HDL-like nanoparticles (also referred to as HDL nanoparticles) mimic natural spherical HDLs in their shape, size, and surface composition (e.g., apolipoprotein A-I, phospholipids). The nanostructures herein may also include a protein such as an apolipoprotein (e.g., apolipoprotein A-I). The nanostructures herein may also be cholesterol-rich (e.g., have a structure comprising cholesterol). The shell may have an inner surface (also referred to as inner leaflet) and an outer surface (also referred to as outer leaflet), such that the therapeutic agent and/or the apolipoprotein may be adsorbed on the outer shell and/or incorporated between the inner surface and outer surface of the shell.

Examples of nanostructures that can be used in the methods are described herein are now described. The structure, nanostructure, or nanoparticle (e.g., a synthetic structure or synthetic nanostructure) has a core and a shell surrounding the core. In embodiments in which the core is a nanostructure, the core includes a surface to which one or more components can be optionally attached. For instance, in some cases, the core is a nanostructure surrounded by a shell, which includes an inner surface and an outer surface. The shell may be formed, at least in part, of one or more components, such as a plurality of lipids, which may optionally associate with one another and/or with surface of the core. For example, components may be associated with the core by being covalently attached to the core, physisorbed, chemisorbed, or attached to the core through ionic interactions, hydrophobic and/or hydrophilic interactions, electrostatic interactions, van der Waals interactions, or combinations thereof. In one particular embodiment, the core includes a gold nanostructure and the shell is attached to the core through a gold-thiol bond.

A number of therapeutic agents are typically associated with the shell of a nanostructure. For instance, at least 20 therapeutic agents may be associated per structure. In general at least 20-30, 20-40, 20-50, 25-30, 25-40, 25-50, 30-40, 30-50, 35-40, 35-50, 40-45, 40-50, 45-50, 50-100, or 30-100 therapeutic agents may be associated per structure.

Optionally, components can be crosslinked to one another. Crosslinking of components of a shell can, for example, allow the control of transport of species into the shell, or between an area exterior to the shell and an area interior of the shell. For example, relatively high amounts of crosslinking may allow certain small, but not large, molecules to pass into or through the shell, whereas relatively low or no crosslinking can allow larger molecules to pass into or through the shell. Additionally, the components forming the shell may be in the form of a monolayer or a multilayer, which can also facilitate or impede the transport or sequestering of molecules. In one exemplary embodiment, the shell includes a lipid bilayer that is arranged to sequester cholesterol and/or control cholesterol efflux out of cells, as described herein.

It should be understood that a shell which surrounds a core need not completely surround the core, although such embodiments may be possible and are contemplated. For example, the shell may surround at least 50%, at least 60%, at least 70%, at least 80%, at least 90%, or at least 99% of the surface area of a core. In some cases, the shell substantially surrounds a core. In other cases, the shell completely surrounds a core. The components of the shell may be distributed evenly across a surface of the core in some cases, and unevenly in other cases. For example, the shell may include portions (e.g., holes) that do not include any material in some cases. If desired, the shell may be designed to allow penetration and/or transport of certain molecules and components into or out of the shell, but may prevent penetration and/or transport of other molecules and components into or out of the shell. The ability of certain molecules to penetrate and/or be transported into and/or across a shell may depend on, for example, the packing density of the components forming the shell and the chemical and physical properties of the components forming the shell. The shell may include one layer of material, or multilayers of materials in some embodiments.

Furthermore, a shell of a structure can have any suitable thickness. For example, the thickness of a shell may be at least 10 Angstroms, at least 0.1 nm, at least 1 nm, at least 2 nm, at least 5 nm, at least 7 nm, at least 10 nm, at least 15 nm, at least 20 nm, at least 30 nm, at least 50 nm, at least 100 nm, or at least 200 nm (e.g., from the innermost surface to the outermost surface of the shell). In some cases, the thickness of a shell is less than 200 nm, less than 100 nm, less than 50 nm, less than 30 nm, less than 20 nm, less than 15 nm, less than 10 nm, less than 7 nm, less than 5 nm, less than 3 nm, less than 2 nm, or less than 1 nm (e.g., from the innermost surface to the outermost surface of the shell). Such thicknesses may be determined prior to or after sequestration of molecules as described herein.

The shell of a structure described herein may comprise any suitable material, such as a hydrophobic material, a hydrophilic material, and/or an amphiphilic material. Although the shell may include one or more inorganic materials such as those listed above for the nanostructure core, in many embodiments the shell includes an organic material such as a lipid or certain polymers. The binding affinity of the nanoparticles may be further altered by including cholesterol (e.g., to modulate fluidity of the lipid monolayer or bilayer).

In one set of embodiments, a structure described herein or a portion thereof, such as a shell of a structure, includes one or more natural or synthetic lipids or lipid analogs (i.e., lipophilic molecules). One or more lipids and/or lipid analogues may form a single layer (e.g., lipid monolayer) or a multi-layer (e.g., a bilayer, lipid bilayer) of a structure. In some instances where multi-layers are formed, the natural or synthetic lipids or lipid analogs interdigitate (e.g., between different layers). Non-limiting examples of natural or synthetic lipids or lipid analogs include fatty acyls, glycerolipids, glycerophospholipids, sphingolipids, saccharolipids and polyketides (derived from condensation of ketoacyl subunits), and sterol lipids and prenol lipids (derived from condensation of isoprene subunits).

In some embodiments, the shell includes a polymer. For example, an amphiphilic polymer may be used. The polymer may be a diblock copolymer, a triblock copolymer, etc . . . , e.g., where one block is a hydrophobic polymer and another block is a hydrophilic polymer. For example, the polymer may be a copolymer of an α-hydroxy acid (e.g., lactic acid) and polyethylene glycol. In some cases, a shell includes a hydrophobic polymer, such as polymers that may include certain acrylics, amides and imides, carbonates, dienes, esters, ethers, fluorocarbons, olefins, styrenes, vinyl acetals, vinyl and vinylidene chlorides, vinyl esters, vinyl ethers and ketones, and vinylpyridine and vinylpyrrolidones polymers. In other cases, a shell includes a hydrophilic polymer, such as polymers including certain acrylics, amines, ethers, styrenes, vinyl acids, and vinyl alcohols. The polymer may be charged or uncharged. As noted herein, the particular components of the shell can be chosen so as to impart certain functionality to the structures.

RNA

There is significant interest in developing synthetic mimics of natural RNA delivery vehicles. In particular, high-density lipoproteins (HDL) are appealing because they naturally bind endogenous RNAs, like microRNA (miRNA), stabilize the single-stranded RNA (ssRNA) to nuclease degradation, and deliver them to target cells to regulate gene expression. HDL-mediated delivery of RNA is dependent upon target cell expression of scavenger receptor type B-1 (also referred to herein as SCARB1 and/or SR-B1). Scavenger receptor class B, type I (SR-BI) is an integral membrane protein found in numerous cell types and tissues, including tissues of the eye. It is a high-affinity receptor for mature, such as the mature HDLs that have apolipoprotein A-I (apoA-I) on their surface. SR-B1 facilitates the uptake of cholesteryl esters from high-density lipoproteins. In addition, SR-B1 is crucial in lipid soluble vitamin uptake. In addition to binding HDL, SR-B1 binds anionic molecules and ligands in a wide variety of sizes.

The terms “microRNA” and “miRNA,” as may be used interchangeably herein, refer to short (e.g., about 20 to about 24 nucleotides in length) non-coding ribonucleic acids (RNAs) that are involved in post-transcriptional regulation of gene expression in multicellular organisms by affecting both the stability and translation of mRNAs. miRNAs are transcribed by RNA polymerase II as part of capped and polyadenylated primary transcripts (pri-miRNAs) that can be either protein-coding or non-coding. The primary transcript is cleaved by the Drosha ribonuclease III enzyme to produce an stem-loop precursor miRNA (pre-miRNA) approximately 70 nucleotides in length, which is further processed in the RNAi pathway. As part of this pathway the pre-miRNA is cleaved by the cytoplasmic Dicer ribonuclease to generate the mature miRNA and antisense miRNA star (miRNA*) products. The mature miRNA is incorporated into an RNA-induced silencing complex (RISC), which recognizes target mRNAs through imperfect base pairing (i.e., partial complementarity) with the miRNA and most commonly results in translational inhibition or destabilization of the target mRNA. This mechanism is most often seen through the binding of the miRNA on the 3′ untranslated region (UTR) of the target mRNA, which can decrease gene expression by either inhibiting translation (for example, by blocking the access of ribosomes for translation) or directly causing degradation of the transcript. The term (i.e., miRNA) may be used herein to any form of the subject miRNA (e.g., precursor, primary, and/or mature miRNA). In some embodiments, the RNA molecule is miRNA. In some embodiments, the miRNA is miR-146a. In some embodiments, the miR-146a has a sequence comprising the sequence of SEQ ID NO: 1. In some embodiments, the miRNA is miR-205. In some embodiments, the miR-205 has a sequence comprising the sequence of SEQ ID NO: 2. In some embodiments, a single nanostructure has two different types of RNA molecules (e.g., miRNAs) complexed to it, wherein the types of RNA molecules have distinct functions (e.g., anti-inflammatory, angiostatic).

Phospholipids

Phospholipids are a class of lipids that comprise hydrophobic fatty acid chains and a hydrophilic head that has a phosphate group and a glycerol molecule. Phospholipids have been widely used to prepare liposomal, ethosomal, and other nanoformulations of topical, oral and parenteral drugs for differing reasons including, but not limited to, improved bio-availability, reduced toxicity and increased permeability across membranes. Naturally occurring phospholipids are fat-like triglycerides containing two long-chained fatty acids and a phosphoric acid radical to which a base is linked. They occur in all animal and vegetable cells, especially in the brain, heart, liver, egg yolk, as well as in soybeans. The most important phospholipids among the naturally occurring phospholipids are the cephalins and lecithins, in which colamine or quoline are present as bases.

Non-limiting examples of phospholipids include, 1,2-Dipalmitoyl-sn-Glycero-3-Phosphothioethanol (DPPTE), phosphatidylcholine, phosphatidylglycerol, lecithin, β, γ-dipalmitoyl-α-lecithin, sphingomyelin, phosphatidylserine, phosphatidic acid, N-(2,3-di(9-(Z)-octadecenyloxy))-prop-1-yl-N,N,N-trimethylammonium chloride, phosphatidylethanolamine, lysolecithin, lysophosphatidylethanolamine, phosphatidylinositol, cephalin, cardiolipin, cerebrosides, dicetylphosphate, dioleoylphosphatidylcholine, dipalmitoylphosphatidylcholine, dipalmitoylphosphatidylglycerol, dioleoylphosphatidylglycerol, palmitoyl-oleoyl-phosphatidylcholine, di-stearoyl-phosphatidylcholine, stearoyl-palmitoyl-phosphatidylcholine, di-palmitoyl-phosphatidylethanolamine, di-stearoyl-phosphatidylethanolamine, di-myrstoyl-phosphatidylserine, di-oleyl-phosphatidylcholine, 1,2-dipalmitoyl-sn-glycero-3-phosphothioethanol (DPPTE), 1,2-dipalmitoyl-sn-glycero-3-phosphoethanolamine-N-[3-(2-pyridyldithio)propionate] (16:0 PDP PE), 1,2-dioleoyl-sn-glycero-3-phosphoethanolamine-N-[3-(2-pyridyldithio)propionate] (18:1 PDP PE), and combinations or derivatives thereof.

Pharmaceutical Compositions

In some embodiments, the disclosure relates to a composition comprising any of the nanostructures as disclosed herein and a pharmaceutically acceptable excipient. As described herein, the “pharmaceutical compositions” or “pharmaceutically acceptable” compositions comprise a therapeutically effective amount of one or more of the structures (e.g., nanostructures) described herein, formulated together with one or more pharmaceutically acceptable excipient (e.g., carriers, additives, and/or diluents). It should be understood that any suitable structures described herein can be used in such pharmaceutical compositions, including those described in connection with the figures. In some cases, the structures in a pharmaceutical composition have a nanostructure core comprising an inorganic material and a shell substantially surrounding and attached to the nanostructure core.

In some embodiments, the pharmaceutical compositions is formulated in liquid or gel form: oral administration, for example, drenches (aqueous or non-aqueous solutions or suspensions), parenteral administration, for example, by subcutaneous, intramuscular, intravenous or epidural injection as, for example, a sterile solution or suspension, or sustained-release formulation; topical application, for example, as a cream, ointment or spray applied to the eye; ocularly or transdermally.

The phrase “pharmaceutically acceptable” is employed herein to refer to those structures, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

In some embodiments, the pharmaceutical compositions of the invention have a pharmaceutically acceptable excipient. Non-limiting examples of pharmaceutically acceptable excipient contemplated include: water, buffered saline, saline, water, lactated ringers solution, cell culture media, serum, dilute serum, creams, polymers, and hydrogels.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

The structures described herein may be orally administered, parenterally administered, subcutaneously administered, and/or intravenously administered. In certain embodiments, a structure or pharmaceutical preparation is administered orally. In other embodiments, the structure or pharmaceutical preparation is administered intravenously. Alternative routes of administration include sublingual, intramuscular, and transdermal administrations.

The amount of active ingredient which can be combined with a carrier material to produce a single dosage form will vary depending upon the host being treated, and the particular mode of administration. The amount of active ingredient that can be combined with a carrier material to produce a single dosage form will generally be that amount of the compound which produces a therapeutic effect. Generally, this amount will range from about 1% to about 99% of active ingredient, from about 5% to about 70%, or from about 10% to about 30%.

Liquid dosage forms for administration of the structures described herein may include pharmaceutically acceptable emulsions, microemulsions, solutions, dispersions, suspensions, syrups, and elixirs. In addition to the inventive structures, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration of a structure described herein include powders, sprays, ointments, pastes, foams, creams, lotions, gels, solutions, patches, drops, and inhalants. The active compound may be mixed under sterile conditions with a pharmaceutically-acceptable carrier, and with any preservatives, buffers, or propellants which may be required.

The ointments, pastes, creams and gels may contain, in addition to the inventive structures, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Ophthalmic formulations contemplated herein include eye ointments, eye drops, powders, solutions, and the like.

Pharmaceutical compositions described herein suitable for parenteral administration comprise one or more inventive structures in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers, which may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the inventive structures may be facilitated by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

When the structures described herein are administered as pharmaceuticals, to humans and animals, they can be given per se or as a pharmaceutical composition containing, for example, about 0.1% to about 99.5%, about 0.5% to about 90%, or the like, of structures in combination with a pharmaceutically acceptable carrier.

The administration may be localized (e.g., to a particular region, physiological system, tissue, organ, or cell type) or systemic, depending on the condition to be treated. For example, the composition may be administered through parental injection, implantation, orally, vaginally, rectally, buccally, pulmonary, topically, nasally, transdermally, surgical administration, or any other method of administration where access to the target by the composition is achieved. Examples of parental modalities that can be used with the invention include intravenous, intradermal, subcutaneous, intracavity, intramuscular, intraperitoneal, epidural, or intrathecal. Examples of implantation modalities include any implantable or injectable drug delivery system. Oral administration may be useful for some treatments because of the convenience to the patient as well as the dosing schedule.

Regardless of the route of administration selected, the structures described herein, which may be used in a suitable hydrated form, and/or the inventive pharmaceutical compositions, are formulated into pharmaceutically-acceptable dosage forms by conventional methods known to those of skill in the art.

The compositions described herein may be given in dosages, e.g., at the maximum amount while avoiding or minimizing any potentially detrimental side effects. The compositions can be administered in effective amounts, alone or in a combinations with other compounds. For example, when treating cancer, a composition may include the structures described herein and a cocktail of other compounds that can be used to treat cancer. When treating conditions associated with abnormal lipid levels, a composition may include the structures described herein and other compounds that can be used to reduce lipid levels (e.g., cholesterol lowering agents).

As used herein, the terms “effective amount” or “therapeutically effective amount” is an amount of nanostructure or composition of the invention, to provide, when administered to a patient, treatment for the disease state or disorder being treated or to otherwise provide the desired effect (e.g., induction of an effective immune response, amelioration of a symptom of the disease). The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art having regard to their knowledge and to this disclosure.

Methods

In preferred embodiments, the nanostructures of the present disclosure are for topical treatments. Current topical therapies for ocular diseases, such as eye drops, ocular ointments, and gels only deliver about 5% of their payload to the anterior ocular chamber and do not readily enter the corneal epithelium. The nanostructures of the present disclosure (e.g., RNA-TLPs) are taken up by cells in the corneal epithelium in vivo. In some embodiments, the nanostructures and/or compositions as described herein are formulated for topical application. In some embodiments, the nanostructures and/or compositions as described herein are topically applied.

Ocular Therapy for Diabetics

In some embodiments, the nanostructures of the present disclosure can be used for the treatment of ocular disorders or ocular disease, such as diabetic keratopathy, in diabetic subjects. Diabetic keratopathy is an ocular complication that occurs with diabetes. In some embodiments, the ocular disorder is diabetic keratopathy. In some embodiments, the ocular disorder is diabetic retinopathy. In some embodiments, the nanostructures and compositions of the instant disclosure are used to treat inflammation. In some embodiments, the nanostructures and compositions of the instant disclosure are used to inhibit NF_{K}B signaling. In some embodiments, the nanostructures and compositions of the instant disclosure are used to treat wounds of the eye. In some embodiments, the wound comprises damage to the epithelium of the cornea. In some embodiments the wound comprises damage to tissues surrounding the epithelium of the cornea.

In some embodiments, the nanostructures and compositions of the instant disclosure are used to treat a subject having an ocular injury or ocular infection. In some embodiments, the ocular disorder, ocular injury or ocular infection is a corneal disorder, corneal injury, or corneal infection, respectively.

Ocular diseases and injuries are particularly difficult to treat in diabetic subjects. The healing process is also very challenging for diabetics after surgeries in which the ocular surface epithelium is compromised (e.g., vitrectomy, cataract extraction). The process of corneal epithelial wound repair, in addition to being lengthened in diabetic subjects, leave them more vulnerable to infection, which can result in irreparable damage. Conventional treatment methods have frequently been ineffective at addressing these issues. They also fail to address the fundamental pathobiology of delayed corneal healing secondary to diabetes.

Application for the Nanostructures

The nanostructure of the present disclosure exhibits increased uptake in the eye compared to other topical eye treatments. Herein, it is shown that RNA-TLPs are taken up by cells in the corneal epithelium in vivo. The HDL-NPs and the RNA-HDL-NPs (e.g., miR-205-HDL-NPs) of the present disclosure are positive agents for healing ocular wounds (e.g., corneal epithelial wounds). Thus, topical treatments (e.g., eye drops, ocular ointments, and gels) containing either HDL-NPs or miR-205-HDL-NPs are contemplated herein. A topical treatment, as contemplated, would be effective for treating wounded corneas (e.g., torn corneal epithelium).

Also contemplated herein is the use of RNA molecules (e.g., miRNAs) with anti-inflammatory properties (e.g., miR-146a) complexed with the HDL-NPs, which would be effective in treating or preventing inflammation (i.e., ocular inflammation) resultant from diseases and injuries of the eye, preferably the corneal epithelial (e.g., dry eye, keratitis, other infections). An effective anti-inflammatory RNA-complexed nanostructure (e.g., miR-HDL-NP) will function as a steroid, without the deleterious side effects that steroids have (e.g., thinning of the cornea, inducing glaucoma).

Also contemplated are RNAs (e.g., miRNAs) with angiostatic properties (e.g., miR-184) complexed with HDL-NPs which are effective in preventing corneal angiogenesis, which can often occur following corneal perturbations.

The present disclosure provides RNAs (e.g., miRNAs) complexed with nanostructures that are exhibit wound healing properties and thus can be used as treatments for diabetic keratopathies (e.g., wound healing), which are not presently available.

Treating

As used herein, the term “treating” refers to partially or completely alleviating, ameliorating, relieving, delaying onset of, inhibiting progression of, reducing severity of, and/or reducing incidence of one or more symptoms or features of a particular disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. In some embodiments, treatment comprises delivery of an inventive targeted particle to a subject.

Subject

As used herein, a “subject” or a “patient” refers to any mammal (e.g., a human), for example, a mammal that may be susceptible to a disease or bodily condition such as a disease or bodily condition that is, for instance, an ocular disease or disorder. Examples of subjects or patients include a human, a non-human primate, a cow, a horse, a pig, a sheep, a goat, a dog, a cat or a rodent such as a mouse, a rat, a hamster, or a guinea pig. A subject may be a subject diagnosed with a certain disease or bodily condition or otherwise known to have a disease or bodily condition. In some embodiments, a subject may be diagnosed as, or known to be, at risk of developing a disease or bodily condition. In certain embodiments, a subject may be selected for treatment on the basis of a known disease or bodily condition in the subject. In some embodiments, a subject may be selected for treatment on the basis of a suspected disease or bodily condition in the subject. In some embodiments, the composition may be administered to prevent the development of a disease or bodily condition. However, in some embodiments, the presence of an existing disease or bodily condition may be suspected, but not yet identified, and a composition of the present invention may be administered to diagnose or prevent further development of the disease or bodily condition.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Synthesis of Gold Nanoparticles (Au NP), Templated Lipoprotein Particles (TLP), and RNA-TLPs

Gold core nanoparticles (Au NPs) are synthesized using standard protocols (Piella et al., 2016). ˜3.5 nm Au seeds are synthesized in by tetrachloroauric acid in excess of sodium citrate and trace amounts of tannic acid to nucleate the Au seeds. Further addition of tetrachloroauric acid and excess sodium citrate results in monodisperse 5 nm Au NP in a seeded growth approach, resulting in a concentration of 70 nM. An aqueous solution of these 5 nm Au NP are mixed with a 5-fold molar excess of purified human apoA-I in a glass vial. The Au NP/apoA-I mixture is incubated for 1 hour at room temperature (RT) on a flat bottom shaker at 60 rpm. Next, 1,2-dipalmitoyl-sn-glycero-3-phosphoethanolamine-N-[3-(2-pyridyldithio)propionate] (PDP-PE; Avanti Polar Lipids) dissolved in chloroform (CHCl₃, 1 mM) or dichloromethane (CH₂Cl₂, 1 mM) is added to the Au NP/apoA-I solution in 250-fold molar excess to the Au NP. The solution is vortexed, followed by addition of 1,2-dioleoyl-sn-glycero-3-phosphocholine (DOPC; Avanti Polar Lipids) or 1:1 solution of cardiolipin (heart, bovine) (CL; Avanti Polar Lipids) and 1,2-dilinoleoyl-sn-glycero-3-phospho-(1′-rac-glycerol) (18:2 PG; Avanti Polar Lipids) dissolved in CHCl₃ (1 mM) or CH₂Cl₂ (1 mM) to the Au NP/apoA-I/PDP-PE solution in 250-fold molar excess to the Au NP and the solution is vortexed. Next, cholesterol dissolved in CHCl₃ (1 mM, Sigma Aldrich) or CH₂Cl₂ is added in 25-fold molar excess to the Au NP. The mixture is vortexed and briefly sonicated (˜2 minutes) causing solution to become opaque and pink in color. The resulting mixture is gradually heated to ˜65° C. with constant stirring to evaporate CHCl₃ or ˜40° C. with constant stirring to evaporate CH₂Cl₂ and to transfer the phospholipids onto the particle surface and into the aqueous phase (˜20 minutes). The reaction is complete when the solution returns to a transparent red color. The resultant TLPs are incubated overnight at RT on a flat bottom shaker at 60 rpm and then purified and concentrated via tangential flow filtration (TFF; KrosFlo Research Iii TFF System, Spectrum Laboratory, model 900-1613). TLPs are stored at 4° C. until use. The concentration of the TLPs is measured using UV-Vis spectroscopy (Agilent 9453) where Au NPs have a characteristic absorption at λₘₐₓ=520 nm, and the extinction coefficient for 5 nm Au NPs is 9.696×10⁶ M⁻¹cm⁻¹.

To synthesize an exemplary RNA-TLP, RNA and 1,2-dioleoyl-3-trimethylammonium-propane (DOTAP) were first mixed. Individual sense and antisense RNA sequences of miR-205, miR-146a, antagomiR-210 or control (Ctrl) (Integrated DNA Technologies) were re-suspended in nuclease free water (500 μM, final). Complement pairs were then mixed in nuclease free water at a concentration enabling direct addition to TLPs (100 nM) at 25-fold molar excess of each RNA sequence (2.5 μM, final per RNA sequence). An ethanolic (EtOH) solution of DOTAP was then added at a 40-fold molar excess to the RNA. The mixture of DOTAP and RNA is briefly sonicated and vortexed (×3) and then incubated at RT for 15 minutes prior to addition to a solution of 100 nM TLPs in water. After the DOTAP-RNA mixture was added to the TLPs, the solvent mixture is 9:1, water:EtOH (v/v). This solution was incubated overnight at RT on a flat bottom shaker at 60 rpm. Resulting RNA-TLPs were purified via centrifugation (15,870×g, 50 min) and the majority of the supernatant with unbound starting materials is removed. The resulting pellet was briefly sonicated back into solution and this material was combined in a single tube as concentrated RNA-TLPs. The concentration of the RNA-TLPs was calculated as described for TLP. For RNA-TLPs, a strong absorption at λₘₐₓ=260 nm confirmed the presence of RNA. For particles synthesized with only one strand of the RNA pair, the synthetic procedure proceeded similarly; however, twice the amount of RNA was added to the TLPs (5 μM, final).

### Example 2: miR-205 HDL-NPs Target SHIP2 in HCECs

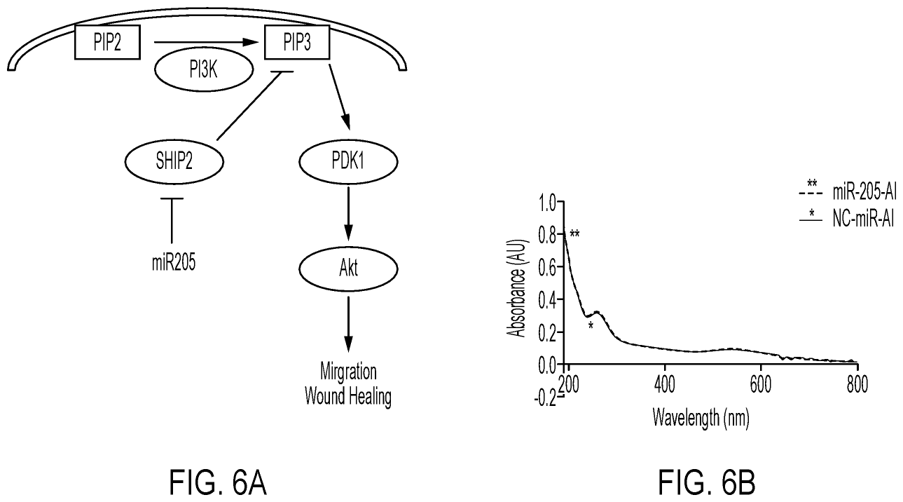

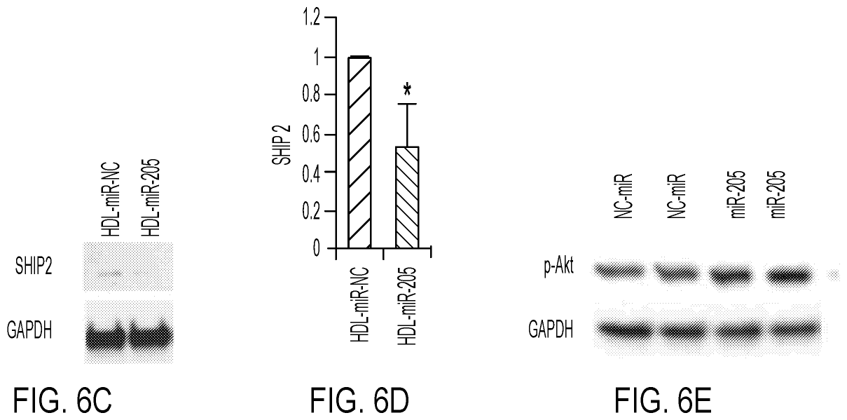

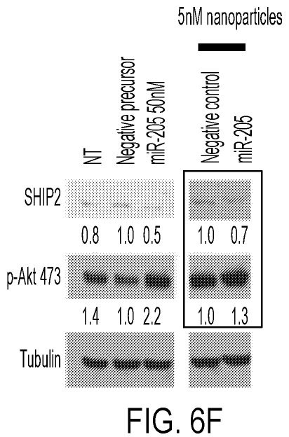

miR-205 negatively regulated the lipid phosphatase SHIP2 in epithelial cells resulting in activation of Akt signaling. SHIP2 limits epithelial cell migration. By suppressing SHIP2, miR-205 promotes epithelial migration via cofilin activation. Herein, a single strand miR-205 mimic was complexed to HDL-NPs and HCECs were exposed to the miR-205-HDL-NP for 48 hrs. Compared with negative particles, miR-205-HDL-NPs decreased SHIP2 and increased p-Akt at 50 nM (FIG. 6F).

### Example 3: miR-205-HDL-NPs Rapidly Seal Scratch Wounds

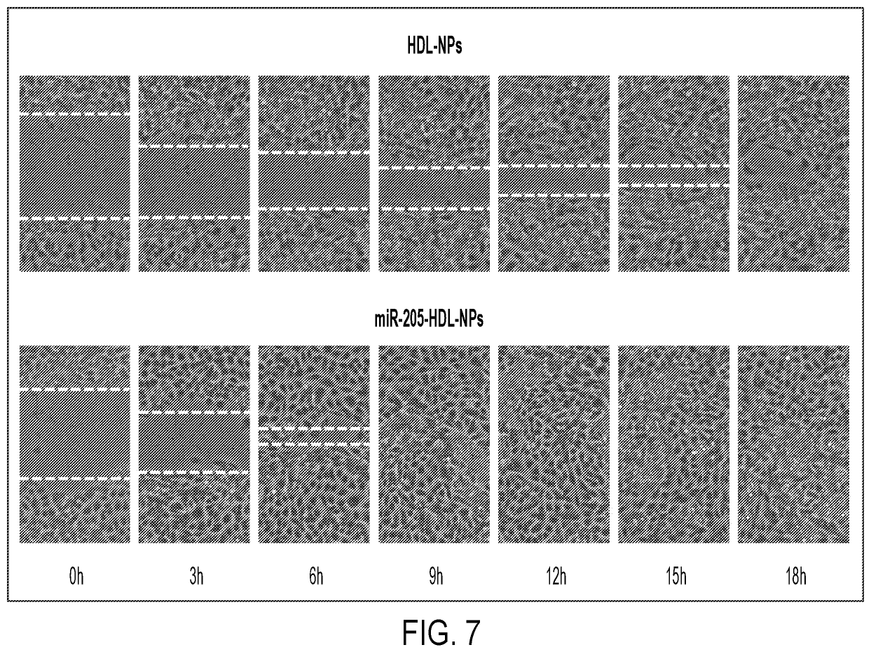

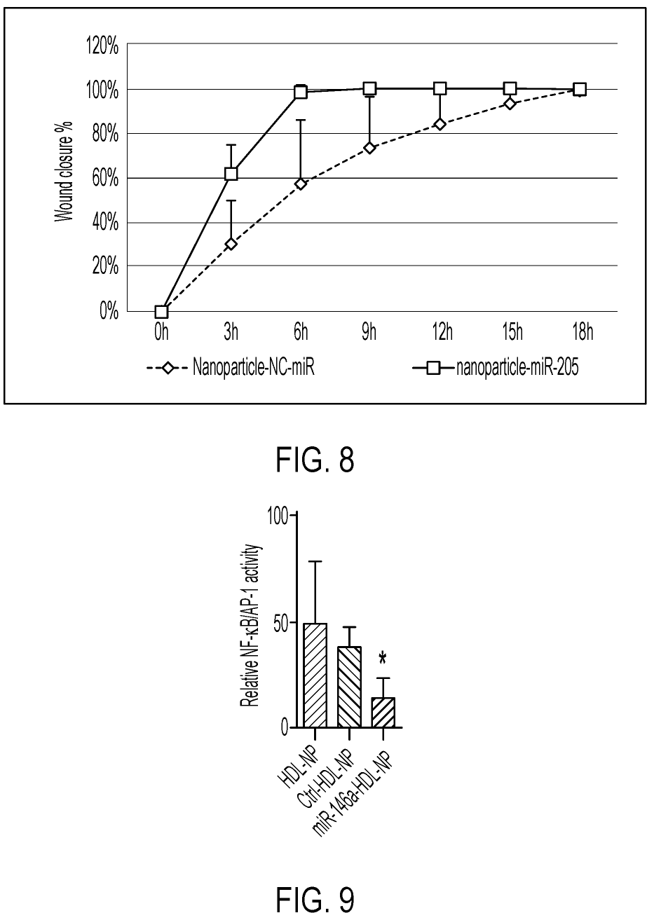

Linear scratch wounds were made to a mitomycin-treated corneal epithelial cell line (hTCEpi) grown to confluence in 0.3 mM Ca+2. Cells were treated with 10 nm solution of control or miR-205 HDL-NPs, imaged and analyzed with a Nikon Biostation. miR-205-HDL-NP-treated hTCEpi cells completely sealed wounds by 6 hours, whereas control HDL-NP-treated hTCEpi cells sealed wounds by 18 hours (FIGS. 7 and 8).

### Example 4: miR-146a-HDL-NPs Reduce NF-κB Activity

miR-146a plays a role in limbal epithelial cell (LEC) maintenance but not in corneal epithelial terminal differentiation. It is upregulated in diabetic LECs and delays cell migration and wound closure in diabetic limbal and corneal epithelial cells. Additionally, it is considered a key gene mediator for proinflammatory signaling regulated by NF-κB. Mouse J774.1 macrophages have the secreted alkaline phosphatase (AP) gene downstream of the NF-κB consensus transcriptional response element.

Herein, a miR-146a mimic was complexed to HDL-NPs and J774.1 murine macrophages were exposed to the miR-146a-HDL-NP (4.5 hrs). After addition of LPS, NF-κB activity was quantified by sampling the cell culture media for secreted AP using a Quant B colorimetric assay. HDL-NPs carrying miR146a significantly reduced the signal of LPS-induced secreted AP (FIG. 9).

### Example 5: Topical Application of HDL-NPs can Penetrate the Unperturbed Ocular Surface

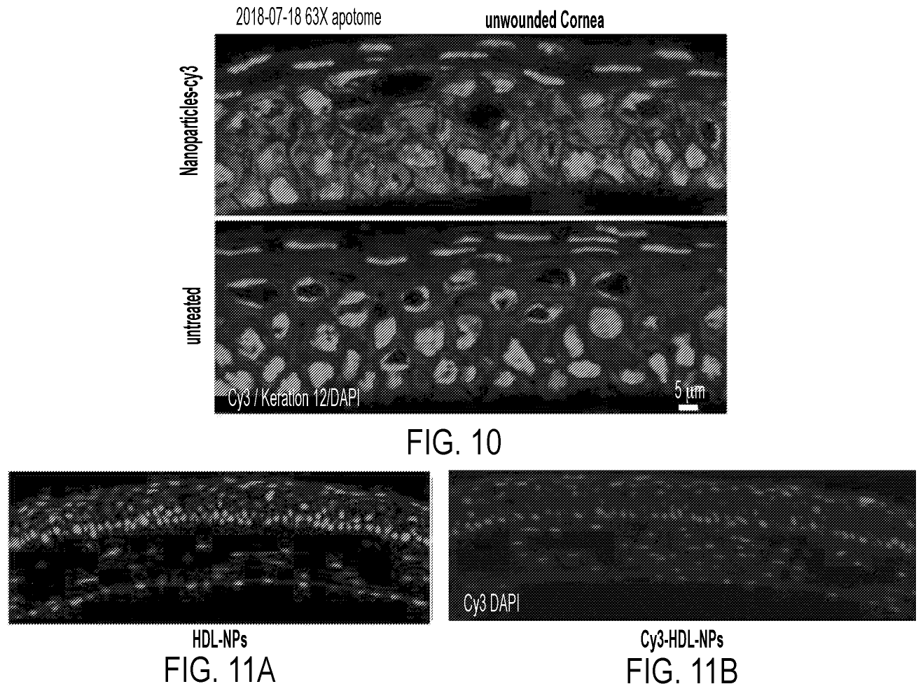

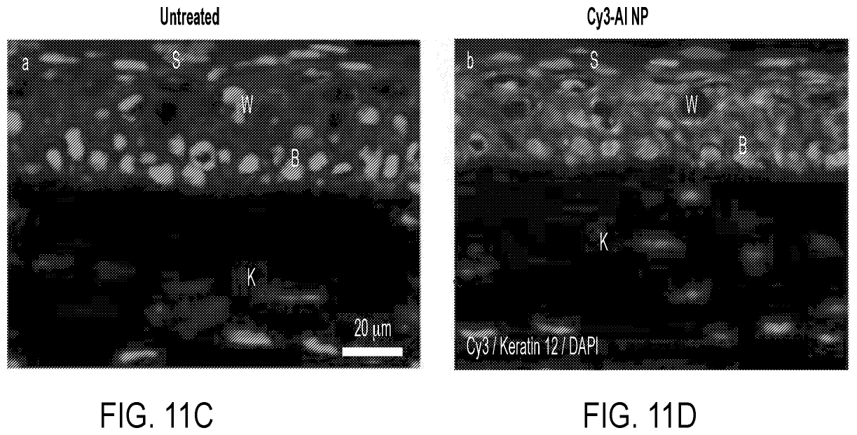

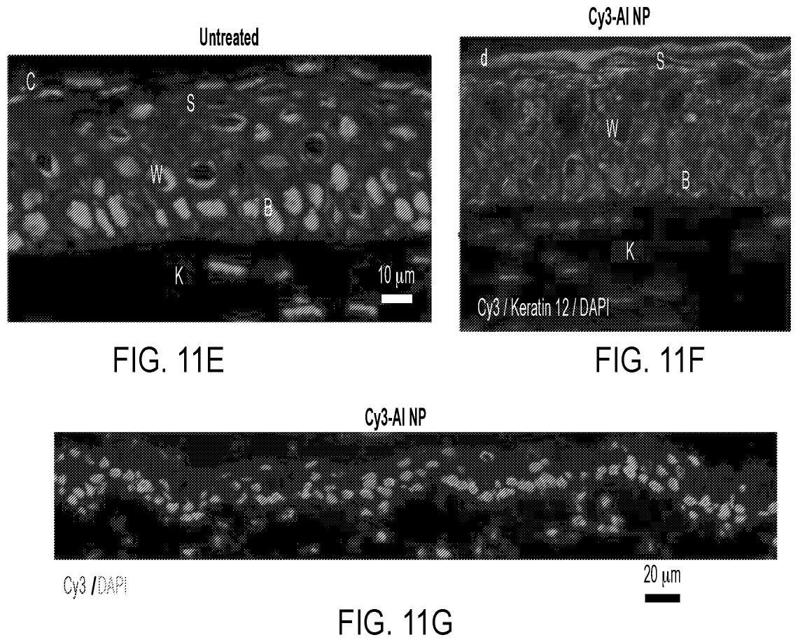

Herein, 3 μl of a Cy-3-tagged HDL-NP (1 μM in PBS) was topically applied to intact non-wounded corneas every 30 minutes for four hours. Twenty-four hours post-treatment, eyes were harvested, embedded in OCT, sectioned and viewed with a fluorescent microscope (FIGS. 10 and 11A-11B).

### Example 6: HDL-NPs and miR-205-HDL-NPs Exhibit Biological Activity In Vivo

miR-205 is a positive regulator of corneal epithelial wound healing, in part, via Akt signaling. HDL contributes to endothelial cell healing by promoting proliferation, migration and ‘tube’ formation via PI3K/Akt signaling. HDL-apoA-I induced angiopoietin like 4 gene in human aortic endothelial cells, which could be blocked by inhibitors of Akt signaling. Since HDL and miR-205 activate the same signaling pathway it is difficult to detect any additive effect of miR-205 via clinical assessment.

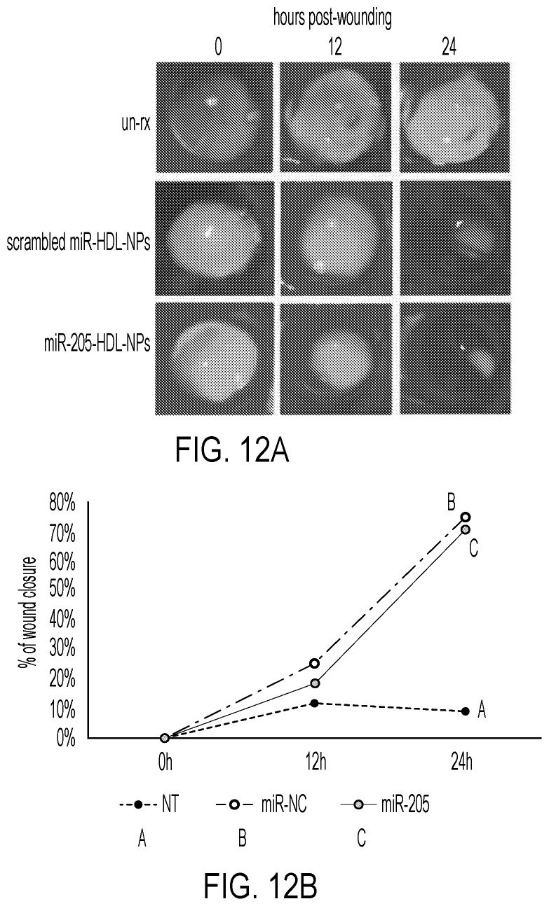

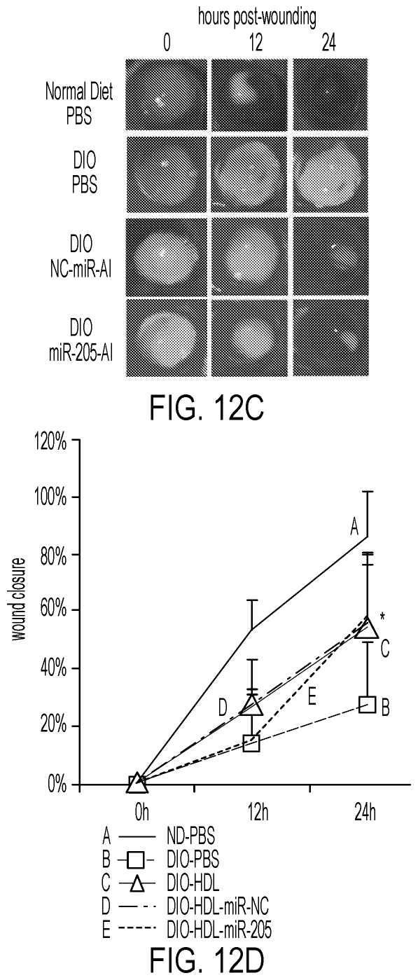

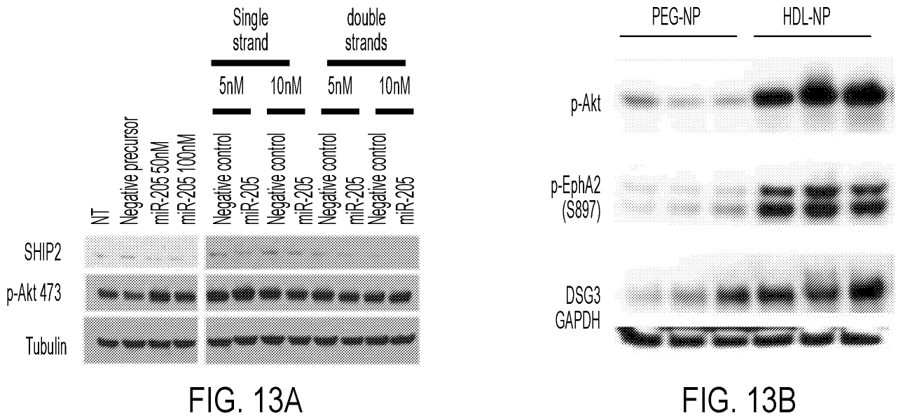

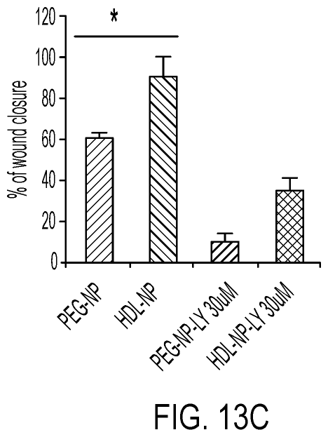

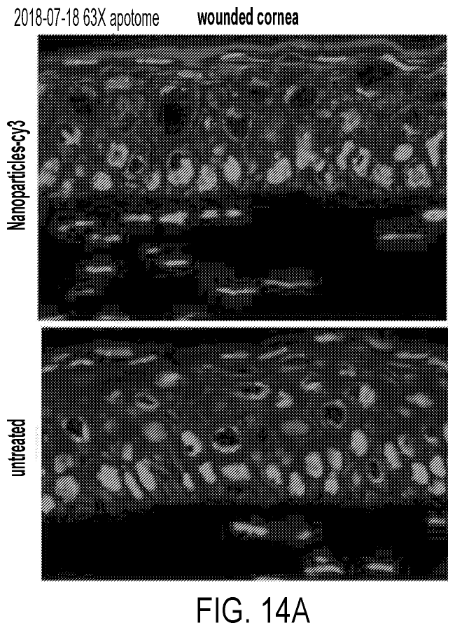

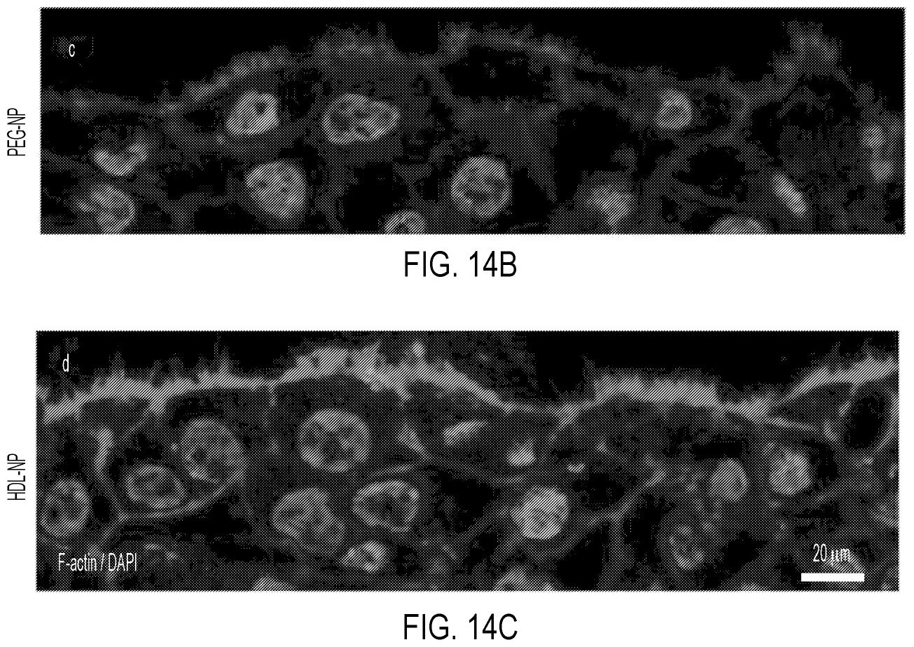

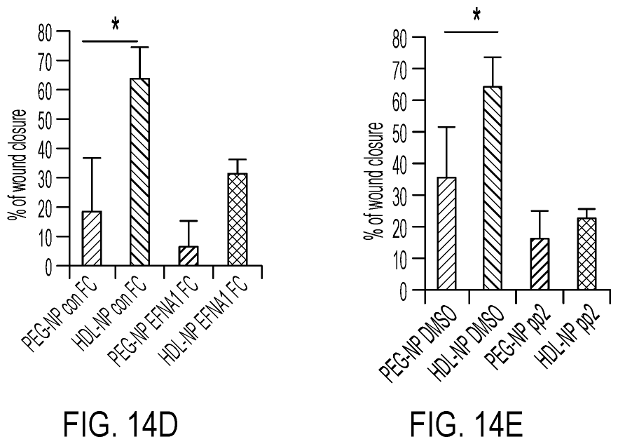

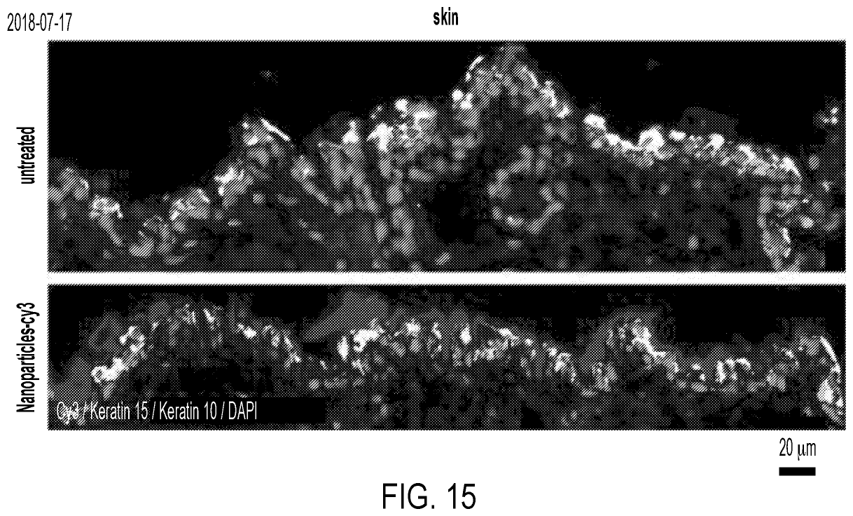

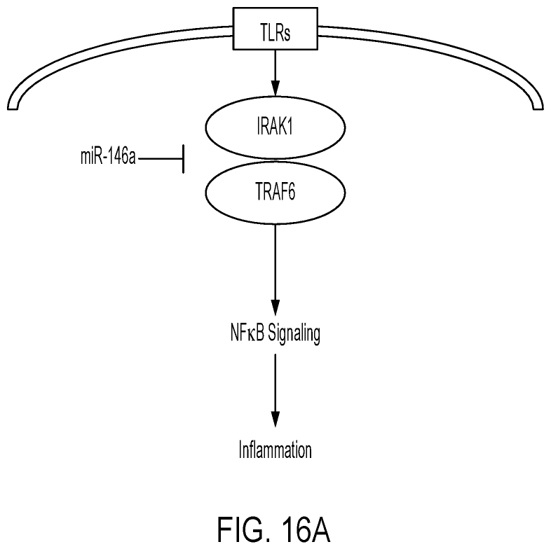

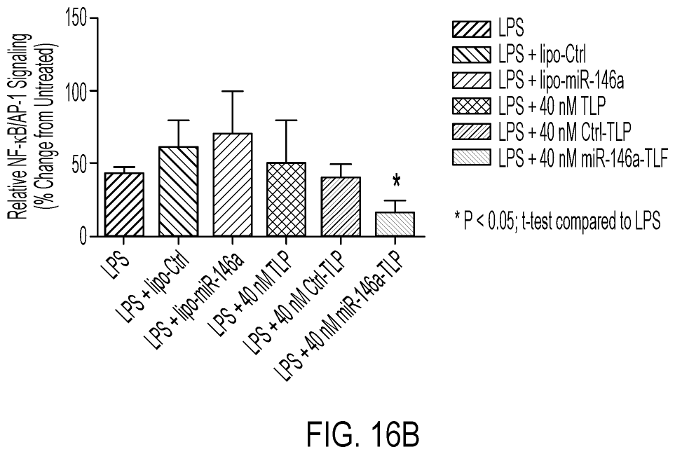

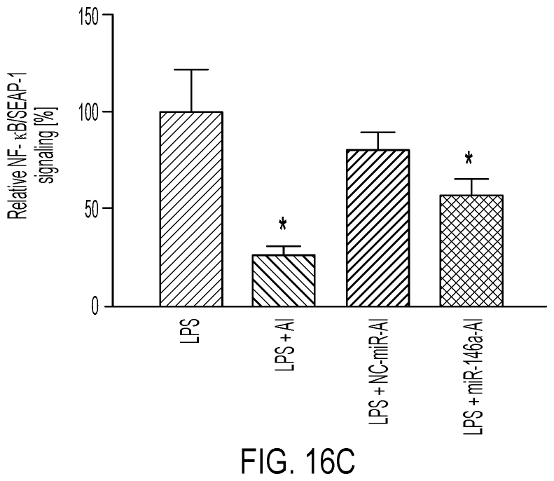

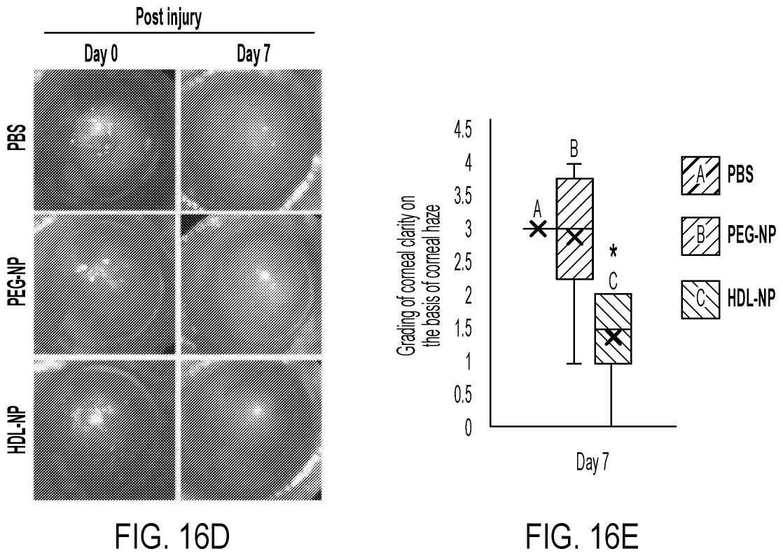

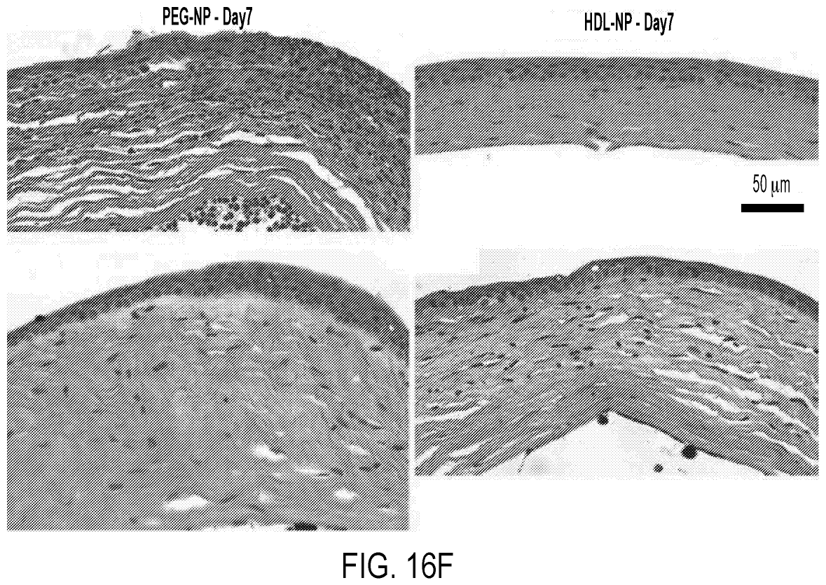

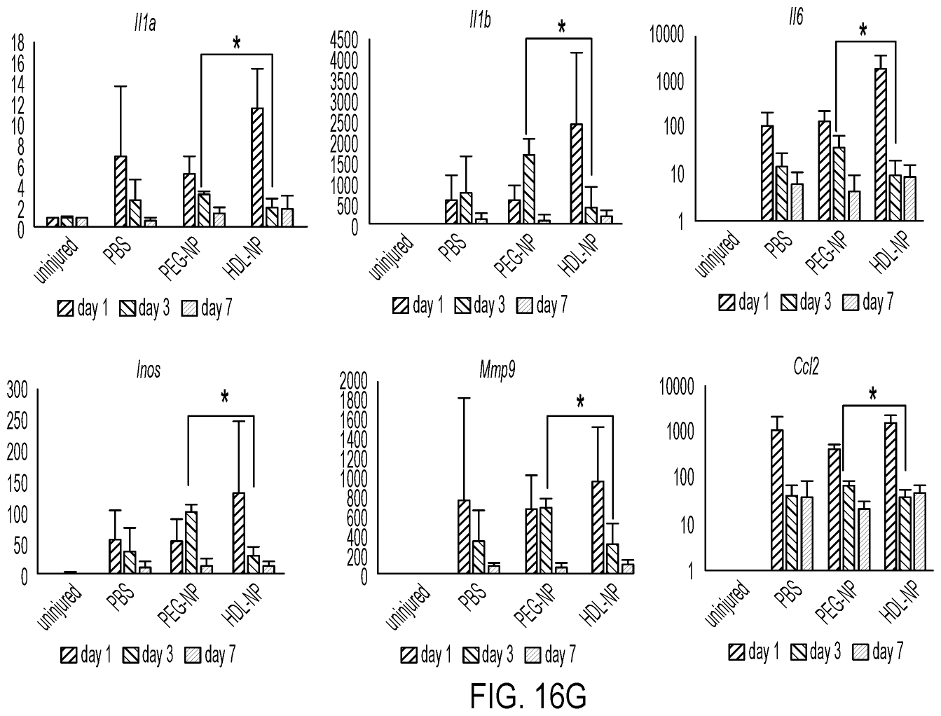

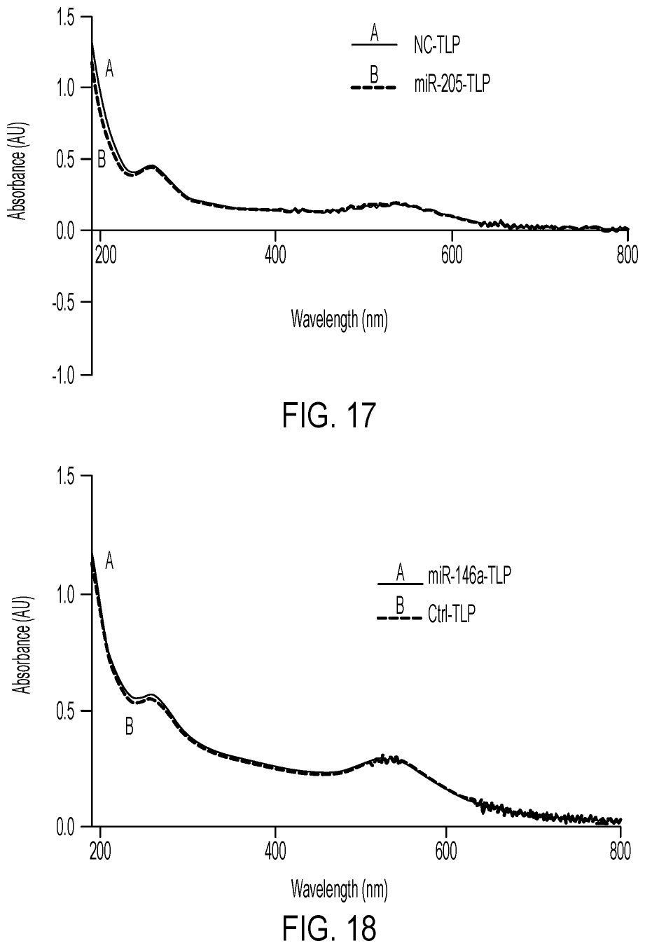

Herein, diet-induced obesity (DIO) mice were anesthetized, and a 1 mm area of central corneal epithelium was removed with a rotating diamond burr. Immediately following wounding, mice (8) received 10 of a miR-205-HDL-NP solution (1 μmole in PBS) or a scrambled miR-HDL-NP solution topically, every 30 minutes for 2 hours. The degree of healing was monitored clinically using a 2% fluorescein stain, and the rate of epithelial healing was evaluated by measuring the wound size with image processing software (ImageJ v.1.5). HDL-NPs and miR-205-HDL-NPs were found to exhibit biological activity in vivo. Both scrambled miR-HDL-NPs and miR-205-HDL-NPs display a positive effect on wound healing (FIGS. 12A-12D).

### Conclusion

Synthetic, functional HDL-NPs can deliver miRNAs to primary human corneal epithelial cells, a macrophage cell line and intact tissues of the limbus/cornea. Both scrambled miR-HDL-NPs and miR-205-HDL-NPs have a positive effect on wound healing in corneal epithelium of diabetic mice. These findings provide a basis for innovative treatment regimens based on miRNA delivery to the corneal surface in normal and diseased situations. One such treatment option is the development of a “super” miRNA-HDL-NP eye treatment (e.g., eye drops) having two miRNAs in order to simultaneously affect biological processes such as angiogenesis and inflammation.

Exemplary Sequences

This Table exhibits some exemplary sequences as disclosed by the instant Specification, but is not limiting. This Specification includes a Sequence Listing submitted concurrently herewith as a text file in ASCII format. The Sequence Listing and all of the information contained therein are expressly incorporated herein and constitute part of the instant Specification as filed.

TABLE 1
Exemplary Sequences

| SEQ | | |
|---|---|---|
| ID | | |
| NO: | Sequence* | Description** |
| 1 | CCGAUGUGUAUCCUCAGCUUUGAGAACUGAAUUCC | miR-146a(NT) |
| | AUGGGUUGUGUCAGUGUCAGACCUCUGAAAUUCAG | |
| | UUCUUCAGCUGGGAUAUCUCUGUCAUCGU | |
| 2 | AAAGAUCCUCAGACAAUCCAUGUGCUUCUCUUGUC | miR-205(NT) |
| | CUUCAUUCCACCGGAGUCUGUCUCAUACCCAACCA | |
| | GAUUUCAGUGGAGUGAAGUUCAGGAGGCAUGGAGC | |
| | UGACA | |

*Unles sotherwise specified, nucleic acid sequences are described 5′ to 3′ and amino acid sequences are described N-terminus to C-terminus.

### OTHER EMBODIMENTS

Embodiment 1. A nanostructure, comprising: a high density lipoprotein nanoparticle (HDL-NP) comprising a core, an apolipoprotein, a lipid shell attached to the core, wherein the lipid shell comprises a phospholipid and an RNA molecule that is associated with the phospholipid.

Embodiment 2. A nanostructure comprising: a templated lipoprotein particle (TLP) comprising a core, an apolipoprotein, a lipid shell attached to the core, wherein the lipid shell comprises a phospholipid and an RNA molecule that is associated with the phospholipid.

Embodiment 3. The nanostructure of any one of embodiments 1-2, wherein the apolipoprotein is apolipoprotein A-I.

Embodiment 4. The nanostructure of any one of embodiments 1-3, further comprising a cholesterol.

Embodiment 5. The nanostructure of any one of embodiments 1-4, wherein the RNA molecule is a microRNA (miRNA).

Embodiment 6. The nanostructure of embodiment 5, wherein the miRNA is miR-205 or miR-146a.

Embodiment 7. A pharmaceutical composition comprising the nanostructure of any one of embodiments 1-6 and a pharmaceutically acceptable excipient.

Embodiment 8. A method of treating a subject having an ocular disorder, comprising: administering the nanostructure of any one of embodiments 1-7 to the subject in an effective amount, thereby treating the ocular disorder.

Embodiment 9. A method of treating a subject having an ocular injury or ocular infection, comprising: administering the nanostructure of any one of embodiments 1-7 to the subject in an effective amount, thereby treating the ocular injury or infection.

Embodiment 10. The method of any one of embodiments 8-9, wherein the ocular disorder, ocular injury or ocular infection is a corneal disorder, corneal injury, or corneal infection, respectively.

Embodiment 11. The method of any one of the embodiments 8-10, wherein the ocular disorder is diabetic keratopathy.

Embodiment 12. The method of any one of embodiments 8-11, wherein the administration is topical.

Embodiment 13. The method of any one of embodiments 8-12, wherein the subject is a mammal.

Embodiment 14. The method of any one of embodiments 8-13, wherein the subject is human.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A nanostructure, comprising:
a high density lipoprotein nanoparticle (HDL-NP) comprising a core, an apolipoprotein, a lipid shell attached to the core, wherein the lipid shell comprises a phospholipid and an RNA molecule that is associated with the phospholipid, wherein the RNA molecule is a microRNA (miRNA).

2. An anionic nanostructure comprising:
an aggregate of cationic lipid-RNA complexes and a templated lipoprotein particle (TLP) wherein the TLP comprises an anionic TLP which is a synthetic HDL having an inert core, a lipid shell surrounding the inert core, and an apolipoprotein functionalized to the inert core, wherein the RNA molecule is a microRNA (miRNA) and wherein the aggregate of cationic lipid-nucleic acid complexes and TLPs forms the anionic nanostructure aggregate.

3. The nanostructure of any one of claims 1-2, wherein the apolipoprotein is apolipoprotein A-I.

4. The nanostructure of any one of claims 1-3, further comprising a cholesterol.

5. The nanostructure of any one of claims 2-4, wherein the cationic lipid-nucleic acid complex is comprised of single stranded miRNA complexed with the cationic lipid.

6. The nanostructure of any one of claims 1-5, wherein the miRNA is miR-205 or miR-146a.

7. The nanostructure of any one of claims 2-6, wherein the aggregate of cationic lipid-nucleic acid complexes and TLPs has a negative ζ-potential.

8. The nanostructure of claim 5, wherein the aggregate of cationic lipid-RNA comprises a mixture of cationic lipid-sense strand RNA and cationic lipid-antisense strand RNA.

9. The nanostructure of any one of claims 1-8, wherein the RNA is not chemically modified.

10. The nanostructure of any one of claims 1-8, wherein the RNA is chemically modified.

11. The nanostructure of any one of claims 1-8, wherein the phospholipids are selected from 1,2-dioleoyl-sn-glycero-3-phophocholine (DOPC) and 1,2-dipalmitoyl-sn-glycero-3-phosphoethanolamine-N-[3-(2-pyridyldithio)propionate] (PDP-PE).

12. The nanostructure of any one of claims 2-8, wherein the nanostructure comprises alternating layers of 1,2-dioleoyl-3-trimethylammonium-propane (DOTAP) and miRNA.

13. A pharmaceutical composition comprising the nanostructure of any one of claims 1-12 and a pharmaceutically acceptable excipient.

14. A method of treating a subject having an ocular disorder, comprising:
administering the nanostructure of any one of claims 1-12 to the subject in an effective amount, thereby treating the ocular disorder.

15. A method of treating a subject having an ocular injury or ocular infection, comprising:
administering the nanostructure of any one of claims 1-12 to the subject in an effective amount, thereby treating the ocular injury or infection.

16. The method of any one of claims 14-15, wherein the ocular disorder, ocular injury or ocular infection is a corneal disorder, corneal injury, or corneal infection, respectively.

17. A method of treating a subject having ocular inflammation, comprising:
administering the nanostructure of any one of claims 1-12 to the subject in an effective amount, thereby treating the ocular inflammation.

18. A method of inhibiting NF_{K}B signaling in a subject having, comprising:
administering the nanostructure of any one of claims 1-2 to the subject in an effective amount, wherein the RNA is miRNA and wherein the miRNA is miR-146a.

19. The method of any one of the claims 14-15, wherein the ocular disorder is diabetic keratopathy.

20. The method of any one of claims 14-19, wherein the administration is topical.

21. The method of any one of claims 14-20, wherein the subject is a mammal.

22. The method of any one of claims 8-21, wherein the subject is human.


SEQUENCE LISTING (2 sequences)

SEQ ID NO: 1 - RNA, 99 nt, Artificial Sequence
  Synthetic Polynucleotide
ccgauguguauccucagcuuugagaacugaauuccauggguugugucagugucagaccucugaaauucaguucuucagcugggauaucucugucaucgu

SEQ ID NO: 2 - RNA, 110 nt, Artificial Sequence
  Synthetic Polynucleotide
aaagauccucagacaauccaugugcuucucuuguccuucauuccaccggagucugucucauacccaaccagauuucaguggagugaaguucaggaggcauggagcugaca